SEQUENCE LISTING (13 sequences)

SEQ ID NO: 1 - protein, 476 aa, synthetic construct
  REGION 1..476: note = Description of Artificial Sequence: Synthetic polypeptide
MRKKRRQRRRPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRKGELNSKLEGKPIPNPLLGLDSTRTGHHHHHH

SEQ ID NO: 2 - protein, 439 aa, Homo sapiens
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 3 - protein, 450 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLR

SEQ ID NO: 4 - protein, 434 aa, Homo sapiens
PLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLR

SEQ ID NO: 5 - protein, 67 aa, Homo sapiens
ELKRSFFALRDQIPELENNEKAPKVVILKKATAYILSVQAEEQKLISEEDLLRKRREQLKHKLEQLR

SEQ ID NO: 6 - protein, 14 aa, unidentified
  REGION 1..14: note = Description of Unknown: E-box DNA binding domain
KRRTHNVLERQRRN

SEQ ID NO: 7 - protein, 10 aa, Human immunodeficiency virus 1
MRKKRRQRRR

SEQ ID NO: 8 - protein, 6 aa, synthetic construct
  REGION 1..6: note = Description of Artificial Sequence: Synthetic 6xHis tag
HHHHHH

SEQ ID NO: 9 - protein, 14 aa, synthetic construct
  REGION 1..14: note = Description of Artificial Sequence: Synthetic peptide
GKPIPNPLLGLDST

SEQ ID NO: 10 - protein, 9 aa, synthetic construct
  REGION 1..9: note = Description of Artificial Sequence: Synthetic peptide
IPNPLLGLD

SEQ ID NO: 11 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 12 - protein, 9 aa, synthetic construct
  REGION 1..9: note = Description of Artificial Sequence: Synthetic peptide
KGELNSKLE

SEQ ID NO: 13 - protein, 9 aa, Human immunodeficiency virus 1
RKKRRQRRR

(19) United States
(12) Patent Application Publication
(10) Pub. No.: US 2025/0381226 A1
(43) Pub. Date: Dec. 18, 2025

(54) COMPOSITIONS AND METHODS FOR TREATING T CELL EXHAUSTION

(71) Applicant: HTYR Acquistion LLC, Philadelphia, PA (US)

(72) Inventors: Yosef Refaeli, Denver, CO (US); Brian Curtis Turner, Denver, CO (US); Thomas R. Payne, Denver, CO (US)

(21) Appl. No.: 19/278,610

(22) Filed: Jul. 23, 2025

Related U.S. Application Data
(62) Division of application No. 17/610,592, filed on Nov. 11, 2021, now Pat. No. 12,370,217.
(60) Provisional application No. 62/847,701, filed on May 14, 2019.

(51) Int. Cl. A61K 35/18; A61K 35/17; A61K 39/00; A61K 39/04; A61K 39/12; A61K 39/39; A61K 40/10; A61K 40/42; A61P 35/00

(52) U.S. Cl. CPC A61K 35/18; A61K 35/17; A61K 39/00; A61K 39/001152; A61K 39/04; A61K 39/12; A61K 39/39; A61K 40/10; A61K 40/4242; A61P 35/00; A61K 2239/31; C12N 2740/16334

(57) ABSTRACT

Provided herein are compositions and methods for treating T cell exhaustion in a subject, by administering a PTD-MYC fusion protein (e.g., an HIV TAT-MYC fusion protein) or immune cells treated with a PTD-MYC fusion protein. Kits for use in practicing the methods are also provided herein.

### CROSS REFERENCE TO RELATED APPLICATIONS

This application is a divisional of U.S. patent application Ser. No. 17/610,592, filed Nov. 11, 2021, which is a 371 national phase filing of International Application No. PCT/US20/32702 filed May 13, 2020, which claims the benefit of priority to U.S. Provisional Patent Application No. 62/847,701, filed May 14, 2019, the entire contents of which are incorporated herein by reference.

### SEQUENCE LISTING INCORPORATION BY REFERENCE

This application contains a Sequence Listing, which has been submitted electronically in xml format and is hereby incorporated by reference in its entirety. Said xml copy, created on Jul. 22, 2025, is named SeqList-365679-00026.xml and is 16,996 bytes in size.

### BACKGROUND OF THE INVENTION

T cell exhaustion is a state of T cell dysfunction that arises during chronic infections and cancer. It is characterized by poor T cell effector function, sustained expression of inhibitory receptors, and a transcriptional state that is distinct from functional effector and memory T cells. Exhaustion negatively affects the immune system's ability to control infection and tumor growth and metastasis.

After an acute infection, naive antigen-specific CD8⁺ T cells become activated, proliferate, acquire effector functions, and differentiate into effector CD8⁺ T cells. Following clearance of the acute infection, most effector CD8⁺ T cells will undergo apoptosis; however, about 5-10% differentiate into memory CD8⁺ T cells. During chronic infection, severe defects in CD8⁺ T cell responses can develop, and antigen-specific CD8⁺ T cells often fail to differentiate into memory CD8⁺ T cells. Loss of effector function (e.g., T-cell exhaustion) occurs in a hierarchical manner, with CD8⁺ T cells progressively losing functions, such as IL-2 production proliferative capacity, and cytotoxicity. Chronic antigen exposure to tumor antigens produces a similar exhaustion phenomenon in CD8⁺ T cells that recognize tumor antigens expressed by cancer cells.

T cell exhaustion can also occur during acute viral infections as well. For example, in certain instances of acute respiratory infections (ARIs), CD8⁺ T cells often exhibit diminished production of cytokines and cytotoxic molecules and exhibit similar patterns of gene expression to that observed in exhausted T cells during chronic infection. In this context, the tendency of CD8⁺ T cells to have significantly reduced functionality in the context of respiratory virus infection is called T cell impairment.

Currently, there is an unmet need for compositions and methods that can decrease and/or reverse T cell exhaustion/impairment and restore effector function after or during a chronic infection and for the treatment of cancer.

### SUMMARY OF THE INVENTION

Provided herein are compositions and methods for treating T cell exhaustion, or T cell impairment, in a subject, by administering a PTD-MYC fusion protein (e.g., an HIV TAT-MYC fusion protein) or immune cells isolated from a donor subject and treated with a PTD-MYC fusion protein. In some embodiments, the T cell exhaustion is associated with a chronic microbial infection (e.g. bacterial, viral, fungal, protozoan or parasitic) or cancer. In some embodiments, the T cell exhaustion, or T cell impairment, is associated with an acute viral infection (e.g. acute respiratory virus infections, such as infection by influenza virus, respiratory syncytial virus (RSV), pneumonia virus, respiratory vaccinia virus, parainfluenza virus, respiratory adenoviruses, severe acute respiratory syndrome corona virus (SARS-CoV), Middle East respiratory syndrome corona virus (MERS-CoV), or human metapneumovirus (HMPV)). In some embodiments, administration of the PTD-MYC fusion protein or PTD-MYC modified immune cells reduces T cell exhaustion, or T cell impairment, in the subject. For example, in some embodiments, administration of the PTD-MYC fusion protein or PTD-MYC modified immune cells reduces the number of exhausted cells (e.g., exhausted T cells) in the subject. In some embodiments, administration of the PTD-MYC fusion protein or PTD-MYC modified immune cells increases the immune response against a pathogen associated with a chronic microbial infection. In some embodiments, administration of the PTD-MYC fusion protein or PTD-MYC modified immune cells alleviate one or more symptoms of a chronic microbial infection.

Provided herein, in certain embodiments, are methods for treating T cell exhaustion, or T cell impairment, in a subject in need thereof, comprising administering an effective amount of one or more modified immune cells to the subject, wherein the one or more modified immune cells comprises a MYC fusion protein comprising (i) a protein transduction domain; and (ii) a MYC polypeptide sequence. In some embodiments, the subject is identified as having altered expression of at least one immune cell marker of T cell exhaustion, or T cell impairment, compared to expression of the at least one immune cell marker in a healthy control. In some embodiments, the immune cell marker is an immune checkpoint protein. In some embodiments, the subject has a microbial infection. In some embodiments, the microbial infection is a chronic microbial infection. In some embodiments, the microbial infection is a bacterial infection, a viral infection, a fungal infection, a protozoan infection, or parasitic infection. In some embodiments, the microbial infection is caused by a pathogen selected from the group consisting of Mycobacterium tuberculosis, Mycobacterium leprae, Mycobacterium bovis, Helicobacter pylori, Staphylococcus aureus, Salmonella Typhi, Treponema pallidum, Escherichia coli, Hemophilus influenza, Pseudomonas aeruginosa, Plasmodium falciparum, Plasmodium vivax, Plasmodium ovale, Plasmodium malariae, Human Immunodeficiency Virus (HIV), Herpesviruses, Herpes Simplex Virus (HSV), Hepatitis B Virus (HBV), Hepatitis C Virus (HCV), Measles Virus, Papovaviruses, Varicella-Zoster Virus, T-Cell Leukemia Viruses, Adenoviruses, Parvoviruses, Epstein-Barr Virus, Enterovirus, Mouse Hepatitis Virus (MHV), Cytomegalovirus (CMV), Papillomaviruses and Lymphocytic Choriomeningitis Virus (LCMV). In some embodiments, the T cell exhaustion, or T cell impairment, is associated with an acute viral infection, such as an acute respiratory virus infection, such as infection by influenza virus, respiratory syncytial virus (RSV), pneumonia virus, respiratory vaccinia virus, parainfluenza virus, respiratory adenoviruses, severe acute respiratory syndrome corona virus (SARS-CoV), Middle East respiratory syndrome corona virus (MERS-CoV), or human metapneumovirus (HMPV)). In some embodiments, the subject has cancer. In some embodiments, the cancer is melanoma. In some embodiments, the melanoma is relapsed refractory melanoma. In some embodiments, the one or more modified immune cells are derived from immune cells isolated from the subject. In some embodiments, the immune cells isolated from the subject are obtained from the peripheral blood of the subject. In some embodiments, the immune cells isolated from the subject are obtained from the lymph node, spleen, or tumor of the subject. In some embodiments, the one or more modified immune cells are prepared by contacting the immune cells in vitro with the MYC fusion protein. In some embodiments, the one or more modified immune cells are prepared by contacting a population of peripheral blood mononuclear cells from the subject in vitro with the MYC fusion protein. In some embodiments, the methods further comprise expanding the modified immune cells in vitro prior to and/or following contacting the modified immune cells with the MYC fusion protein. In some embodiments, the protein transduction domain sequence is a TAT protein transduction domain sequence. In some embodiments, the MYC fusion protein comprises SEQ ID NO: 1. In some embodiments, the MYC fusion protein translocates to the nucleus of one or more modified immune cells. In some embodiments, the MYC fusion protein exhibits a biological activity of MYC. In some embodiments, the altered expression comprises an increase in the cell surface expression of one or more immune cell receptors. In some embodiments, the one or more cell surface receptors comprises PD-1, LAG-3, CD160, 2B4, or any combination thereof. In some embodiments, the one or more modified immune cells are administered intravenously, intraperitoneally, subcutaneously, intramuscularly, or intratumorally. In some embodiments, the subject is a human or a non-human animal. In some embodiments, the one or more modified immune cells comprises one or more T cells. In some embodiments, the one or more modified immune cells comprises one or more CD8⁺ T cells. In some embodiments, the one or more modified immune cells comprises one or more exhausted immune cells. In some embodiments, the one or more modified immune cells comprises one or more exhausted T cells. In some embodiments, the one or more modified immune cells comprises one or more exhausted CD8⁺ T cells.

Provided herein, in certain embodiments, are methods for treating a chronic microbial infection in a subject in need thereof, comprising administering an effective amount of one or more modified immune cells to the subject, wherein the one or more modified immune cells comprise a MYC fusion protein comprising (i) a protein transduction domain; and (ii) a MYC polypeptide sequence. In some embodiments, the subject is identified as having altered expression of at least one immune cell marker of T cell exhaustion, or T cell impairment, compared to expression of the at least one immune cell marker in a healthy control. In some embodiments, the immune cell marker is an immune checkpoint protein. In some embodiments, the microbial infection is a bacterial infection, a viral infection, a fungal infection, a protozoan infection, or parasitic infection. In some embodiments, the microbial infection is caused by a pathogen selected from the group consisting of Mycobacterium tuberculosis, Mycobacterium leprae, Mycobacterium bovis, Helicobacter pylori, Staphylococcus aureus, Salmonella Typhi, Treponema pallidum, Escherichia coli, Hemophilus influenza, Pseudomonas aeruginosa, Plasmodium falciparum, Plasmodium vivax, Plasmodium ovale, Plasmodium malariae, Human Immunodeficiency Virus (HIV), Herpesviruses, Herpes Simplex Virus (HSV), Hepatitis B Virus (HBV), Hepatitis C Virus (HCV), Measles Virus, Papovaviruses, Varicella-Zoster Virus, T-Cell Leukemia Viruses, Adenoviruses, Parvoviruses, Epstein-Barr Virus, Enterovirus, Mouse Hepatitis Virus (MHV), Cytomegalovirus (CMV), Papillomaviruses and Lymphocytic Choriomeningitis Virus (LCMV). In some embodiments, the subject was previously vaccinated against the pathogen. In some embodiments, the one or more modified immune cells are derived from immune cells isolated from the subject. In some embodiments, the immune cells isolated from the subject are obtained from the peripheral blood of the subject. In some embodiments, the immune cells isolated from the subject are obtained from the lymph node, spleen, or tumor of the subject. In some embodiments, the one or more modified immune cells are prepared by contacting the T cells in vitro with the MYC fusion protein. In some embodiments, the methods further comprise expanding the cells in vitro prior to and/or following contacting the cells with the MYC fusion protein. In some embodiments, the protein transduction domain sequence is a TAT protein transduction domain sequence. In some embodiments, the MYC fusion protein comprises SEQ ID NO: 1. In some embodiments, the MYC fusion protein translocates to the nucleus of one or more immune cells in the immune cell population. In some embodiments, the MYC fusion protein exhibits a biological activity of MYC. In some embodiments, the altered expression comprises an increase in the cell surface expression of one or more immune cell receptors. In some embodiments, the one or more cell surface receptors comprises PD-1, LAG-3, CD160, 2B4, or any combination thereof. In some embodiments, the one or more modified immune cells are administered intravenously, intraperitoneally, subcutaneously, intramuscularly, or intratumorally. In some embodiments, the subject is a human or a non-human animal. In some embodiments, the one or more modified immune cells comprises one or more T cells. In some embodiments, the one or more modified immune cells comprises one or more CD8⁺ T cells. In some embodiments, the one or more modified immune cells comprises one or more exhausted immune cells. In some embodiments, the one or more modified immune cells comprises one or more exhausted T cells. In some embodiments, the one or more modified immune cells comprises one or more exhausted CD8⁺ T cells.

Provided herein, in certain embodiments, are methods for treating an acute respiratory infection associated with T cell impairment in a subject in need thereof, comprising administering an effective amount of one or more modified immune cells to the subject, wherein the one or more modified immune cells comprise a MYC fusion protein comprising (i) a protein transduction domain; and (ii) a MYC polypeptide sequence. In some embodiments, the subject is identified as having altered expression of at least one immune cell marker of T cell exhaustion, or T cell impairment, compared to expression of the at least one immune cell marker in a healthy control. In some embodiments, the immune cell marker is an immune checkpoint protein. In some embodiments, the acute respiratory virus infection is caused by a pathogen selected from the group consisting of influenza virus, respiratory syncytial virus (RSV), pneumonia virus, respiratory vaccinia virus, parainfluenza virus, respiratory adenoviruses, severe acute respiratory syndrome corona virus (SARS-CoV), Middle East respiratory syndrome corona virus (MERS-CoV), and human metapneumovirus (HMPV)). In some embodiments, the subject was previously vaccinated against the pathogen. In some embodiments, the one or more modified immune cells are derived from immune cells isolated from the subject. In some embodiments, the immune cells isolated from the subject are obtained from the peripheral blood of the subject. In some embodiments, the immune cells isolated from the subject are obtained from the lymph node, spleen, or tumor of the subject. In some embodiments, the one or more modified immune cells are prepared by contacting the T cells in vitro with the MYC fusion protein. In some embodiments, the methods further comprise expanding the cells in vitro prior to and/or following contacting the cells with the MYC fusion protein. In some embodiments, the protein transduction domain sequence is a TAT protein transduction domain sequence. In some embodiments, the MYC fusion protein comprises SEQ ID NO: 1. In some embodiments, the MYC fusion protein translocates to the nucleus of one or more immune cells in the immune cell population. In some embodiments, the MYC fusion protein exhibits a biological activity of MYC. In some embodiments, the altered expression comprises an increase in the cell surface expression of one or more immune cell receptors. In some embodiments, the one or more cell surface receptors comprises PD-1, LAG-3, CD160, 2B4, or any combination thereof. In some embodiments, the one or more modified immune cells are administered intravenously, intraperitoneally, subcutaneously, intramuscularly, or intratumorally. In some embodiments, the subject is a human or a non-human animal. In some embodiments, the one or more modified immune cells comprises one or more T cells. In some embodiments, the one or more modified immune cells comprises one or more CD8⁺ T cells. In some embodiments, the one or more modified immune cells comprises one or more exhausted immune cells. In some embodiments, the one or more modified immune cells comprises one or more exhausted T cells. In some embodiments, the one or more modified immune cells comprises one or more exhausted CD8⁺ T cells.

Also provided herein, in certain embodiments, are uses of one or more modified immune cells for treating T cell exhaustion, or T cell impairment, in a subject in need thereof, wherein the one or more modified immune cells comprise a MYC fusion protein comprising (i) a protein transduction domain; and (ii) a MYC polypeptide sequence.

Also provided herein, in certain embodiments, are uses of one or more modified immune cells for treating a chronic microbial infection or acute respiratory virus infection associated with T cell exhaustion, or T cell impairment, in a subject in need thereof, wherein the one or more modified immune cells comprise a MYC fusion protein comprising (i) a protein transduction domain; and (ii) a MYC polypeptide sequence.

### BRIEF DESCRIPTION OF THE DRAWINGS

FIGS. 1A-1C illustrate the level of CD86 expression in populations of immune cells isolated from donor subjects, following pre-treatment with the PTD-MYC fusion polypeptide or vehicle control, and activation under various conditions.

FIGS. 2A-2E illustrate the level of CD279 (PD-I) expression (FIGS. 2A-2C) and CD152 (CTLA-4) expression (FIGS. 2A and 2D-2E) in populations of immune cells isolated from donor subjects, following pre-treatment with the PTD-MYC fusion polypeptide or vehicle control, and activation under various conditions.

FIGS. 3A-3E illustrate the level of CD274 (PD-LI) expression (FIGS. 3A-3C) and CD25 (IL2RA) expression (FIGS. 3A and 3D-3E) in populations of immune cells isolated from donor subjects, following pre-treatment with the PTD-MYC fusion polypeptide or vehicle control, and activation under various conditions.

FIGS. 4A-4C illustrate the level of CD279 (PD-I) and CD152 (CTLA-4) expression in peripheral blood mononuclear cells (PBMCs) isolated from patients enrolled in a relapsed refractory melanoma clinical study. FIG. 4A shows flow cytometry histograms of PBMC samples labeled with fluorescent antibodies to CD279 that were collected from two relapsed refractory melanoma patients (101 and 103) before and after a one-hour treatment with Tat-MYC protein (TBX-3400). FIG. 4B is a set of flow cytometry diagrams showing intracellular staining results for two different tags on the Tat-MYC protein (His or VS) in PBMCs isolated from a patient enrolled in a relapsed refractory melanoma trial. The patient's PBMCs were either left untreated (left panels) or treated with Tat-MYC (TBX-4000) for one hour (right panels). FIG. 4C is a set of charts showing the results of a flow cytometry analysis of PBMC samples treated with Tat-MYC (TBX-4000) for one hour in a closed system. Samples were analyzed for intracellular levels of Tat-MYC by using fluorescent antibodies that bind to two different Tat-MYC tags (the His Tag and the VS tag). The left-hand chart shows the results of cells stained for the protein on day zero (Od) and on the following day (Id). In the right-hand chart, Tat-MYC treated cells (TBX-3400; (+)) and untreated cells (PBMC-No Treatment; (−)) from the patient were activated with antibodies to CD3 and CD28. The expression levels of T cell activation marker, CD25, and the checkpoint markers, CD279 and CD152, were analyzed by flow cytometry.

### DETAILED DESCRIPTION OF THE INVENTION

It is to be appreciated that certain aspects, modes, embodiments, variations, and features of the present technology are described below in various levels of detail in order to provide a substantial understanding of the present technology.

### I. Definitions

The definitions of certain terms as used in this specification are provided below. Unless defined otherwise, all technical and scientific terms used herein generally have the same meaning as commonly understood by one of ordinary skill in the art to which this present technology belongs.

As used herein, the singular forms “a”, “an”, and “the” are intended to include the plural forms as well, unless the context clearly indicates otherwise.

As used herein, the term “about” means that a value can vary+/−20%, +/−15%, +/−10% or+/−5% and remain within the scope of the present disclosure. For example, “a concentration of about 200 IU/mL” encompasses a concentration between 160 IU/mL and 240 IU/mL.

As used herein, the term “administration” of an agent to a subject includes any route of introducing or delivering the agent to a subject to perform its intended function. Administration can be carried out by any suitable route, including intravenously, intramuscularly, intraperitoneally, or subcutaneously. Administration includes self-administration and the administration by another.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form and a second plurality are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

The terms “polypeptide,” “peptide,” and “protein” are used interchangeably herein to refer to a polymer of amino acid residues. The terms apply to naturally occurring amino acid polymers as well as amino acid polymers in which one or more amino acid residues is a non-naturally occurring amino acid, e.g., an amino acid analog. The terms encompass amino acid chains of any length, including full length proteins, wherein the amino acid residues are linked by covalent peptide bonds.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a composition known to exhibit the desired therapeutic effect) and a negative control (a subject or a sample that does not receive the therapy or receives a placebo) are typically employed.

As used herein, the term “effective amount” or “therapeutically effective amount” refers to a quantity of an agent sufficient to achieve a desired therapeutic effect. In the context of therapeutic applications, the amount of a therapeutic protein administered to the subject can depend on the type and severity of the infection and on the characteristics of the individual, such as general health, age, sex, body weight and tolerance to drugs. It can also depend on the degree, severity and type of disease. The skilled artisan will be able to determine appropriate dosages depending on these and other factors.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell.

The term “linker” refers to synthetic sequences (e.g., amino acid sequences) that connect or link two sequences, e.g., that link two polypeptide domains. In some embodiments, the linker contains 1, 2, 3, 4, 5, 6, 7, 8, 9, or 10 of amino acid sequences.

As used herein the term immune cell refers to any cell that plays a role in the immune response. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes.

The term “lymphocyte” refers to all immature, mature, undifferentiated and differentiated white lymphocyte populations including tissue specific and specialized varieties. It encompasses, by way of non-limiting example, B cells, T cells, NKT cells, and NK cells. In some embodiments, lymphocytes include all B cell lineages including pre-B cells, progenitor B cells, early pro-B cells, late pro-B cells, large pre-B cells, small pre-B cells, immature B cells, mature B cells, plasma B cells, memory B cells, B-1 cells, and B-2 cell populations.

As used herein, the term T-cell includes naive T cells, CD4+ T cells, CD8⁺ T cells, memory T cells, activated T cells, exhausted T cells, tolerant T cells, chimeric T cells, and antigen-specific T cells.

The term “B cell” or “B cells” refers to, by way of non-limiting example, a pre-B cell, progenitor B cell, early pro-B cell, late pro-B cell, large pre-B cell, small pre-B cell, immature B cell, mature B cell, naive B cells, plasma B cells, activated B cells, exhausted B cells, tolerant B cells, chimeric B cells, antigen-specific B cells, memory B cell, B-1 cell, and B-2 cell populations.

As used herein “adoptive cell therapeutic composition” refers to any composition comprising cells suitable for adoptive cell transfer. In exemplary embodiments, the adoptive cell therapeutic composition comprises a cell type selected from a group consisting of a tumor infiltrating lymphocyte (TIL), TCR (i.e. heterologous T-cell receptor) modified lymphocytes and CAR (i.e. chimeric antigen receptor) modified lymphocytes. In another embodiment, the adoptive cell therapeutic composition comprises a cell type selected from a group consisting of T-cells, exhausted T-cells, CD8+ cells, CD4+ cells, NK-cells, delta-gamma T-cells, and regulatory T-cells. In another embodiment, TILs, T-cells, CD8+ cells, CD4+ cells, NK-cells, delta-gamma T-cells, regulatory T-cells or peripheral blood mononuclear cells form the adoptive cell therapeutic composition. In one embodiment, the adoptive cell therapeutic composition comprises T cells. In one embodiment, the adoptive cell therapeutic composition may be a composition comprising one or more primary immune cells isolated from a donor subject which have been contacted with a PTD-MYC fusion protein, comprising (i) a protein transduction domain; (ii) a MYC polypeptide sequence.

As used herein, the term “exhausted immune cell,” “exhausted T cell,” and “exhausted B cell” refer to dysfunctional T cells and B cells, and does not encompass anergic immune cells, anergic T cells, or anergic B cells. Exhausted immune cells, exhausted T cells, and exhausted B cells are characterized by progressive loss of effector functions during chronic infections or cancer with some functions that are exhausted early (e.g., IL-2, cytotoxicity, and proliferation), whereas others (e.g., IFN-γ) persist longer. Anergic immune cells, anergic T cells, or anergic B cells can arise when immune cells receive initial TCR signals in the absence of co-stimulation, leading to a state of hyporesponsiveness. Notably, anergy seems to be a state of nonresponsiveness that is molecularly distinct from exhaustion (Wherry, J. E. Nature Immunology 12:492-499 (2011); Wherry, J. E. et al. Immunity 27:670-684 (2007)).

The terms “MYC” and “MYC gene” are synonyms. They refer to a nucleic acid sequence that encodes a MYC polypeptide. A MYC gene comprises a nucleotide sequence of at least 120 nucleotides that is at least 60% to 100% identical or homologous, e.g., at least 60, 65%, 70%, 75%, 80%, 85%, 86%, 87%, 88%, 90%, 91%, 92%, 94%, 95%, 96%, 97%, 98%, or any other percent from about 70% to about 100% identical to sequences of NCBI Accession Number NM-002467. In some embodiments, the MYC gene is a proto-oncogene. In certain instances, a MYC gene is found on chromosome 8, at 8q24.21. In certain instances, a MYC gene begins at 128,816,862 bp from pter and ends at 128,822,856 bp from pter. In certain instances, a MYC gene is about 6 kb. In certain instances, a MYC gene encodes at least eight separate mRNA sequences-5 alternatively spliced variants and 3 unspliced variants.

The terms “MYC protein,” “MYC polypeptide,” and “MYC sequence” are synonyms and refer to the polymer of amino acid residues disclosed in NCBI Accession Number UniProtKB/Swiss-Prot: P01106.1 (MYC isoform 1) orNP_002458.2 (UniProtKB/Swiss-Prot: P01106.2; MYC isoform 2), and functional homologs, analogs or fragments thereof. The sequence of or UniProtKB/Swiss-Prot: P01106.1 is:

(SEQ ID NO: 2)
mplnvsftnrnydldydsvqpyfycdeeenfyqqqqqselqppa
psediwkkfellptpplspsrrsglcspsyvavtpfslrgdndg
gggsfstadqlemvtellggdmvnqsficdpddetfikniiiqd
cmwsgfsaaaklvseklasyqaarkdsgspnparghsvcstssl
ylqdlsaaasecidpsvvfpyplndssspkscasqdssafspss
dsllsstesspqgspeplvlheetppttssdseeeqedeeeidv
vsvekrqapgkrsesgspsagghskpphsplvlkrchvsthqhn
yaappstrkdypaakrvkldsvrvlrqisnnrkctsprssdtee
nvkrrthnvlerqrrnelkrsffalrdqipelennekapkvvil
kkatayilsvqaeeqkliseedllrkrreqlkhkleqlrnsca

The sequence of NP_002458.2 (UniProtKB/Swiss-Prot: P01106.2) is:

(SEQ ID NO: 11)
mdffrvvenqqppatmplnysftnrnydldydsvqpyfycdeee
nfyqqqqqselqppapsediwkkfellptpplspsrrsglcsps
yvavtpfslrgdndggggsfstadqlemytellggdmvnqsfic
dpddetfikniiiqdcmwsgfsaaaklvseklasyqaarkdsgs
pnparghsvcstsslylqdlsaaasecidpsyvfpyplndsssp
kscasqdssafspssdsllsstesspqgspeplvlheetpptts
sdseeeqedeeeidyysvekrqapgkrsesgspsagghskpphs
plylkrchysthqhnyaappstrkdypaakrvkldsvrvlrqis
nnrkctsprssdteenykrrthnvlerqrrnelkrsffalrdqi
pelennekapkvvilkkatayilsvqaeeqkliseedllrkrre
qlkhkleqlrnsca

In some embodiments, the MYC polypeptide is a complete MYC polypeptide sequence. In some embodiments, the MYC polypeptide is a partial MYC polypeptide sequence. In some embodiments, the MYC polypeptide comprises at least 400 consecutive amino acids of SEQ ID NO: 2 OR 11. In some embodiments, the MYC polypeptide comprises at least 400 consecutive amino acids of SEQ ID NO: 2 OR 11 and retains at least one MYC activity. In some embodiments, the MYC polypeptide comprises at least 400, at least 410, at least 420, at least 430, or at least 450 consecutive amino acids of SEQ ID NO: 2 OR 11. In some embodiments, the MYC polypeptide comprises at least 400, at least 410, at least 420, at least 430, or at least 450 consecutive amino acids of SEQ ID NO: 2 OR 11 and retains at least one MYC activity. In some embodiments, the MYC polypeptide is c-MYC. In some embodiments, the MYC polypeptide sequence comprises the sequence shown below:

(SEQ ID NO: 3)
MDFFRVVENQQPPATMPLNVSFTNRNYDLDYDSVQPYFYCDEEE
NFYQQQQQSELQPPAPSEDIWKKFELLPTPPLSPSRRSGLCSPS
YVAVTPFSLRGDNDGGGGSFSTADQLEMVTELLGGDMVNQSFIC
DPDDETFIKNIIIQDCMWSGFSAAAKLVSEKLASYQAARKDSGS
PNPARGHSVCSTSSLYLQDLSAAASECIDPSVVFPYPLNDSSSP
KSCASQDSSAFSPSSDSLLSSTESSPQGSPEPLVLREETPPTTS
SDSEEEQEDEEELDVVSVEKRQAPGKRSESGSPSAGGHSKPPHS
PLVLKRCHVSTHQHNYAAPPSTRKDYPAAKRVKLDSVRVLRQIS
NNRKCTSPRSSDTEENVKRRTHNVLERQRRNELKRSFFALRDQI
PELENNEKAPKVVILKKATAYILSVQAEEQKLISEEDLLRKRRE
QLKIAKLEQLR.

In some embodiments, the MYC polypeptide sequence comprises the sequence shown below:

(SEQ ID NO: 4)
PLNVSFTNRNYDLDYDSVQPYFYCDEEENFYQQQQQSELQPPAP
SEDIWKKFELLPTPPLSPSRRSGLCSPSYVAVTPFSLRGDNDGG
GGSFSTADQLEMVTELLGGDMVNQSFICDPDDETFIKNIIIQDC
MWSGFSAAAKLVSEKLASYQAARKDSGSPNPARGHSVCSTSSLY
LQDLSAAASECIDPSVVFPYPLNDSSSPKSCASQDSSAFSPSSD
SLLSSTESSPQGSPEPLVLREETPPTTSSDSEEEQEDEEEIDVV
SVEKRQAPGKRSESGSPSAGGHSKPPHSPLVLKRCHVSTHQHNY
AAPPSTRKDYPAAKRVKLDSVRVLRQISNNRKCTSPRSSDTEEN
VKRRTHNVLERQRRNELKRSFFALRDQIPELENNEKAPKVVILK
KATAYILSVQAEEQKLISEEDLLRKRREQLKIAKLEQLR.

In some embodiments, a MYC polypeptide comprises an amino acid sequence that is at least 40% to 100% identical, e.g., at least 40%, 45%, 50%, 55%, 60%, 65%, 70%, 75%, 80%, 81%, 82%, 83%, 84%, 85%, 86%, 87%, 88%, 90%, 91%, 92%, 94%, 95%, 96%, 97%, 98%, 99%, or any other percent from about 40% to about 100% identical to the sequence of NCBI Accession Number NP002458.2 or UniProtKB/Swiss-Prot Accession Number POI 106.1. In some embodiments, MYC polypeptide refers to a polymer of 439 amino acids, a MYC polypeptide that has not undergone any post-translational modifications. In some embodiments, MYC polypeptide refers to a polymer of 439 amino acids that has undergone post-translational modifications. In some embodiments, the MYC polypeptide is 48,804 kDa. In some embodiments, the MYC polypeptide contains a basic Helix-Loop-Helix Leucine Zipper (bHLH/LZ) domain. In some embodiments, the bHLH/LZ domain comprises the sequence of: ELKRSFF ALRDQIPELENNEKAPKVVILKKATAYILSVQAEEQKLISEEDLLRKRREQLKH KLEQLR (SEQ ID NO: 5). In some embodiments, the MYC polypeptide is a transcription factor (e.g., Transcription Factor 64). In some embodiments, the MYC polypeptide contains an E-box DNA binding domain. In some embodiments, the MYC polypeptide binds to a sequence comprising CACGTG. In some embodiments, the MYC polypeptide promotes one or more of cell survival and/or proliferation. In some embodiments, a MYC polypeptide includes one or more of those described above, and includes one or more post-translational modifications (e.g., acetylation). In some embodiments, the MYC polypeptides comprise one or more additional amino acid residues at the N-terminus or C-terminus of the polypeptide. In some embodiments, the MYC polypeptides are fusion proteins. In some embodiments, the MYC polypeptides are linked to one or more additional peptides at the N-terminus or C-terminus of the polypeptide.

Proteins suitable for use in the methods described herein also includes functional variants, including proteins having between 1 to 15 amino acid changes, e.g., 1, 2, 3, 4, 5, 6, 7, 8, 9, 10, 11, 12, 13, 14, or 15 amino acid substitutions, deletions, or additions, compared to the amino acid sequence of any protein described herein. In other embodiments, the altered amino acid sequence is at least 75% identical, e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any protein inhibitor described herein. Such sequence-variant proteins are suitable for the methods described herein as long as the altered amino acid sequence retains sufficient biological activity to be functional in the compositions and methods described herein. Where amino acid substitutions are made, the substitutions can be conservative amino acid substitutions. Among the common, naturally occurring amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff et al. (1992), Proc. Natl Acad Sci. USA, 89:10915-10919). Accordingly, the BLOSUM62 substitution frequencies are used to define conservative amino acid substitutions that, in some embodiments, are introduced into the amino acid sequences described or disclosed herein. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

The phrases “E-box sequence” and “enhancer box sequence” are used interchangeably herein and mean the nucleotide sequence CANNTG, wherein N is any nucleotide. In certain instances, the E-box sequence comprises CACGTG. In certain instances, the basic helix-loop-helix domain of a transcription factor encoded by MYC binds to the E-box sequence. In certain instances, the E-box sequence is located upstream of a gene (e.g., p21, Be1-2, or ornithine decarboxylase). In certain instances, the MYC polypeptide contains an E-box DNA binding domain. In certain instances, the E-box DNA binding domain comprises the sequence of KRRTHNVLERQRRN (SEQ ID NO: 6). In certain instances, the binding of the transcription factor encoded by MYC to the E-box sequence, allows RNA polymerase to transcribe the gene downstream of the E-box sequence.

The term “MYC activity” or “MYC biological activity” or “biologically active MYC” or “biological activity of MYC” includes one or more of enhancing or inducing cell survival, cell proliferation, and/or antibody production. By way of example and not by way of limitation, MYC activity includes enhancement of expansion of anti-CD3 and anti-CD28 activated T-cells and/or increased proliferation of long-term self-renewing hematopoietic stem cells. MYC activity also includes entry into the nucleus of a cell, binding to a nucleic acid sequence (e.g., binding an E-box sequence), and/or inducing expression of MYC target genes.

The terms “patient,” “subject,” “individual,” and the like are used interchangeably herein, and refer to an animal, typically a mammal. In one embodiment, the patient, subject, or individual is a mammal. In one embodiment, the patient, subject or individual is a human. In some embodiments the patient, subject or individual is an animal, such as, but not limited to, domesticated animals, such as equine, bovine, murine, ovine, canine, and feline.

The terms “protein transduction domain (PTD)” or “transporter peptide sequence” (also known as cell permeable proteins (CPP) or membrane translocating sequences (MTS)) are used interchangeably herein to refer to small peptides that are able to ferry much larger molecules into cells independent of classical endocytosis. In some embodiments, a nuclear localization signal can be found within the protein transduction domain, which mediates further translocation of the molecules into the cell nucleus.

The terms “treating” or “treatment” as used herein covers the treatment of a disease in a subject, such as a human, and includes: (i) inhibiting a disease, i.e., arresting its development; (ii) relieving a disease, i.e., causing regression of the disease; (iii) slowing progression of the disease; and/or (iv) inhibiting, relieving, or slowing progression of one or more symptoms of the disease. With respect to a tumor, “treating” or “treatment” also encompasses regression of a tumor, slowing tumor growth, inhibiting metastasis of a tumor, inhibiting relapse or recurrent cancer and/or maintaining remission.

It is also to be appreciated that the various modes of treatment or prevention of medical diseases and conditions as described are intended to mean “substantial,” which includes total but also less than total treatment or prevention, and wherein some biologically or medically relevant result is achieved. The treatment can be a continuous prolonged treatment for a chronic disease or a single, or few time administrations for the treatment of an acute condition.

The term “therapeutic” as used herein means a treatment and/or prophylaxis. A therapeutic effect is obtained by suppression, remission, or eradication of a disease state.

### II. Overview

The present disclosure relates to the treatment of T cell exhaustion, or T cell impairment, in a subject using a MYC fusion protein comprising a protein transduction domain and a MYC polypeptide sequence. In certain embodiments, the T cell exhaustion, or T cell impairment, results from chronic conditions, such as chronic viral infection or cancer. In certain embodiments, the T cell exhaustion, or T cell impairment, results from acute respiratory viral infection. In some embodiments, T cell exhaustion, or T cell impairment, occurs following vaccination. In some embodiments, T cell exhaustion, or T cell impairment, occurs during active infection in an individual that has been previously vaccinated.

The present disclosure is based, at least in part, on the discovery, that contacting T cells isolated from a donor subject with a PTD-MYC fusion polypeptide containing a MYC polypeptide and a protein transduction domain (PTD), such as the HIV TAT protein transduction domain, advantageously results in a decrease in the expression of cell surface immune checkpoint proteins, including but not limited to, Programmed cell death protein I (PD-1), also known as CD279 (cluster of differentiation 279), or cytotoxic T-lymphocyte-associated protein 4 (CTLA-4), also known as CD152 (cluster of differentiation 152). PD-I and CTLA-4 are inhibitory receptors that promote T cell exhaustion during chronic microbial infection and in cancer. Additional co-receptors, such as LAG-3, CD244 (2B4), CD160, TIM-3 also contribute to this effect. Blocking receptor signaling from these receptors, for example, by using inhibitory antibodies that target and block these receptors, has been shown to substantially decrease T cell dysfunction and increase cytotoxic T cell responses. It is found herein that similar effects can be achieved using a MYC fusion protein to downregulate these receptors, thus decreasing the negative signaling pathways that result in T cell exhaustion, or T cell impairment. In some embodiments, a MYC fusion protein provided herein is employed to reverse immune cell exhaustion/impairment. In some embodiments, a MYC fusion protein provided herein is employed to prevent or ameliorate immune cell exhaustion/impairment.

In one aspect, the present disclosure provides a method for treating or preventing T cell exhaustion, or T cell impairment, in a subject in need thereof, wherein the method comprises administering an effective amount of one or more modified immune cells (e.g., T cells, such as, for example, CD8⁺ T cells) to the subject, wherein the one or more modified immune cells comprise a PTD-MYC fusion protein comprising (i) a protein transduction domain; and (ii) a MYC polypeptide sequence. In some embodiments, the subject is identified as having altered expression of at least one or more immune cell markers associated with T cell exhaustion, or T cell impairment, compared to that observed in a healthy control. In some embodiments, the one or more modified immune cells are derived from immune cells isolated from the subject. In some embodiments, immune cells are isolated from an allogenic donor. The immune cells can be obtained from the peripheral blood, lymph node, spleen, or a tumor. In some embodiments, the immune cells comprise one or more lymphocytes. In some embodiments, the one or more lymphocytes comprise a T cell, a B cell, an NK cell, or any combination thereof. In some embodiments, the one or more lymphocytes comprise a T cell. In some embodiments, the one or more lymphocytes comprise a CD8⁺ T cell. In some embodiments, the one or more lymphocytes comprise one or more exhausted lymphocytes from the subject (e.g. one or more exhausted T cells, for example, one or more exhausted CD8⁺ T cells). In some embodiments, the one or more lymphocytes do not comprise exhausted lymphocytes but are isolated from a subject having one or more exhausted lymphocytes.

In some embodiments, the one or more modified immune cells may be prepared by contacting a population of immune cells (e.g., CD8⁺ T cells) in vitro with the MYC fusion protein following isolation from the subject. In some embodiments, the method may further include expanding the immune cells in vitro prior to contacting the cells with the MYC fusion protein. In some embodiments, the method may further include expanding the immune cells in vitro following contacting the primary immune cells with the MYC fusion protein.

In another aspect, the present disclosure provides a method for treating or preventing T cell exhaustion, or T cell impairment, in a subject in need thereof, wherein the method comprises administering an effective amount of a MYC fusion protein comprising (i) a protein transduction domain; and (ii) a MYC polypeptide sequence. In some embodiments, the subject is identified as having altered expression of at least one or more immune cell markers associated with T cell exhaustion, or T cell impairment, compared to that observed in a healthy control.

In some embodiments, the MYC fusion protein or one or more modified immune cells comprising the MYC fusion protein may be administered by any appropriate method, e.g., intravenously, intraperitoneally, subcutaneously, intramuscularly, or intratumorally.

Exemplary PTD-MYC fusion proteins for use in the methods are provided herein. The PTD-MYC fusion proteins provided are able to enter immune cells (e.g., T cell) and translocate to the nucleus, where the fusion protein can activate one or more MYC-responsive genes. In some embodiments, the protein transduction domain sequence of the MYC fusion protein comprises a TAT protein transduction domain sequence. In some embodiments, the MYC polypeptide portion of the MYC fusion protein comprises the amino sequence set forth in SEQ ID NO: 2 or 11. In some embodiments, the PTD-MYC fusion protein comprises the amino sequence set forth in SEQ ID NO: 1.

In some embodiments, subjects for treatment with a MYC fusion protein provided herein or a modified immune cell comprising a MYC fusion protein provided herein exhibit increased expression one or more immune cell markers, wherein increased or sustained expression of the immune cell marker is associated with T cell exhaustion, or T cell impairment, compared to that observed in a healthy control (e.g., a non-exhausted T cell). In some embodiments, the one or more immune cell markers that are increased is an immune checkpoint protein. In some embodiments, the one or more immune cell markers that are increased is selected from the group consisting of PD1, LAG3, CD160, 2B4 (CD244) and a combination of any two or more thereof.

In some embodiments, the one or more immune cell markers that are increased is selected from the group consisting of PD-1, CTLA-4, LAG-3, CD160, 2B4, IL-7ra, IL-15ra, KLRG-1, TIM-3, Eomes and a combination of any two or more thereof.

In some embodiments, the one or more immune cell markers that are increased in an exhausted T cell is selected from the group consisting of CD244, PDCD1, CTLA4, GP49B, PTGER4, CD160, LAG3, IL7R, IL15RA, HAVCR2, PTGER2, CD7, TNFRSF9, GLYCOP, VCAM1, TNFSF6, ITM2A, MOX2, ITGAV, CD9,CCL3, CXCR4, CCL4, CCRL2, SMAD1, RGS16, GPR56, TANK, DUSP1, GPR65, PTPN13, PRKWNK, IL6ST, ITPR5, JAK.3, MAP3K1, SH2D2A, SOCS3, ACTN1, ISG20, G1P2, ICSBP1, SERPINA, TCRG-V4, TCRB-V1, PBX3, EOMES, ATF1, AHR, EGR2, HFATC1, ZFP91, HIST1H2, BCL2, CASP3, GAS2, CKS2, CAR2, PLSCR1, SLC12A2, ART3, GPD2, NDUFA5, CYP4V3, ENTPD1, P2RX4, C76628, SFRS7, MTV43, C79248, PENK1, COCH, NR4A2, A43010, IER5, SEPTIN, RCN, WBP5, PBEF1, PPM1B, DDIT4, KDT1, TRIM47, HRB, RNF1, NRIP1, TUBB2, SPP1, KLK6, LY75, SCL29A1, BUB1, NDFIP1, SPRED2, CBX4, and a combination of any two or more thereof.

In some embodiments, treatment with a MYC fusion protein provided herein or a modified immune cell comprising a MYC fusion protein results in a decrease of at least one or more immune cell markers selected from the group consisting of CD244, PDCD1, CTLA4, GP49B, PTGER4, CD160, LAG3, IL7R, IL15RA, HAVCR2, PTGER2, CD7, TNFRSF9, GLYCOP, VCAM1, TNFSF6, ITM2A, MOX2, ITGAV, CD9,CCL3, CXCR4, CCL4, CCRL2, SMAD1, RGS16, GPR56, TANK, DUSP1, GPR65, PTPN13, PRKWNK, IL6ST, ITPR5, JAK.3, MAP3K1, SH2D2A, SOCS3, ACTN1, ISG20, GIP2, ICSBP1, SERPINA, TCRG-V4, TCRB-V1, PBX3, EOMES, ATF1, AHR, EGR2, HFATC1, ZFP91, HIST1H2, BCL2, CASP3, GAS2, CKS2, CAR2, PLSCR1, SLC12A2, ART3, GPD2, NDUFA5, CYP4V3, ENTPD1, P2RX4, C76628, SFRS7, MTV43, C79248, PENK1, COCH, NR4A2, A43010, IER5, SEPTIN, RCN, WBP5, PBEF1, PPM1B, DDIT4, KDT1, TRIM47, HRB, RNF11, NRIP1, TUBB2, SPP1, KLK6, LY75, SCL29A1, BUB1, NDFIP1, SPRED2, CBX4, and a combination of any two or more thereof, compared to that observed prior to administration.

In some embodiments, subjects for treatment with a MYC fusion protein provided herein or a modified immune cell comprising a MYC fusion protein provided herein exhibit decreased expression one or more immune cell markers, where decreased expression of the immune cell marker is associated with T cell exhaustion, or T cell impairment, compared to that observed in a healthy control (e.g., a non-exhausted T cell). In some embodiments, the one or more immune cell markers that is decreased is selected from the group consisting of IGSFI0, SELPL, H2-Q8, FCGR2B, CD3D, ITGB2, ITGAI, ITGA4, KLRK1, SEMA4A, IL18RI, KLRDI, LY6C, KLRCI, EDG6, ITGB7, ITGAX, KLRGI, CKLFSF7, LSPI, CCR5, CCR2, H2-K1, H2-D1, H2-Q10, H2-Q7, SIAT10, LGALSI0, RAC2, PIK3CD, ILK, MAP3K8, ARHGEFI, SI00A6, STK38, LCK, ARHGAPI, SI00A4, RASA3, JAKI, SI00AI, HCPH, DGKA, PTK9L, IL18RAP, MAPILC3, ACTB, IFNGR, TPP2, LYZS, CAPN2, CTSD, USP3, PRSS19, MUS, BZWI, RORA, KLF2, RUNXI, HOD, KLF3, SATB1, BNIP3L, ANXA2, ANXA6, ANXAI, VAMP3, SNX5, SNXI0, CDC37, CDKN2D, PAK2, CDC34, HMGCSI, RPN2, NDUFB6, PDHAI, ARL6IP5, PSMD13, FXYD5, ARL2BP, UBEIX, APOBEC, KCTDI0, SSR3, UGCG, MGC68, GARS, SDHA, HSPA8, RRM2, ATPSL, PFKP, HBB-B1, HBB-A1, TCEB2, EIF3S8, RPS16, EIF2S1, SFPQ, RPLI0A, RPL36, EEF2, RPS7, PIGT, RPL27A, PPGB, TAGLN2, HINTI, LEFI, LSPI, TOBI, SDCBP, SEP15, MTVR2, GABARAPL2, PLD3, ETSI, DSTN, LBR, GOLPH2, WDRI, PLP2, NME2, KCNAB2, DIAPI, TXNDC5, SMFN, STARDI0, CLICI, TMSBI0, IAP, GM2A, ATP6V0B, DNAJDI, SUPT4H, PTPRC, CRIPI, EMPI, PLAC8, and a combination of any two or more thereof.

In some embodiments, treatment with a MYC fusion protein provided herein or a modified immune cell comprising a MYC fusion protein results in an increase of at least one or more immune cell markers selected from the group consisting of IGSF I 0, SELPL, H2-Q8,FCGR2B, CD3D, ITGB2, ITGAI, ITGA4, KLRK1, SEMA4A, IL18R1, KLRDI, LY6C, KLRCI, EDG6, ITGB7, ITGAX, KLRGI, CKLFSF7, LSPI, CCR5, CCR2, H2-K1, H2-D1, H2-QI0, H2-Q7, SIAT10, LGALSI0, RAC2, PIK3CD, ILK, MAP3K8, ARHGEFI, SI00A6, STK38, LCK, ARHGAPI, SI00A4, RASA3, JAKI, SI00AI, HCPH, DGKA, PTK9L, IL18RAP, MAPILC3, ACTB, IFNGR, TPP2, LYZS, CAPN2, CTSD, USP3, PRSS19, MUS, BZWI, RORA, KLF2, RUNXI, HOD, KLF3, SATBI, BNIP3L, ANXA2, ANXA6, ANXAI, VAMP3, SNX5, SNXI0, CDC37, CDKN2D, PAK2, CDC34, HMGCSI, RPN2, NDUFB6, PDHAI, ARL6IP5, PSMD13, FXYD5, ARL2BP, UBEIX, APOBEC, KCTDI0, SSR3, UGCG, MGC68, GARS, SDHA, HSPA8, RRM2, ATPSL, PFKP, HBB-B1, HBB-A1, TCEB2, EIF3S8, RPS16, EIF2S1, SFPQ, RPLI0A, RPL36, EEF2, RPS7, PIGT, RPL27A, PPGB, TAGLN2, HINTI, LEFI, LSPI, TOBI, SDCBP, SEP15, MTVR2, GABARAPL2, PLD3, ETSI, DSTN, LBR, GOLPH2, WDR1, PLP2, NME2, KCNAB2, DIAP1, TXNDC5, SMFN, STARDI0, CLIC1, TMSBI0, IAP, GM2A, ATP6V0B, DNAJD1, SUPT4H, PTPRC, CRIP1, EMP1, PLAC8, and a combination of any two or more thereof, compared to that observed prior to administration. In some embodiments, treatment with a MYC fusion protein provided herein or a modified immune cell comprising a MYC fusion protein results in an increase in cytokine production in the subject compared to that observed prior to administration. In some embodiments, treatment with a MYC fusion protein provided herein or a modified immune cell comprising a MYC fusion protein results in an increase in production of IL-2, TNF, Granzyme B, and/or IFN gamma in the subject compared to that observed prior to administration.

In another aspect, the present disclosure provides a method for treating a chronic microbial infection in a subject in need thereof, wherein the method comprises administering an effective amount of one or more modified immune cells (e.g., CD8⁺ T cells) to the subject, wherein the one or more modified immune cells comprise a PTD-MYC fusion protein comprising (i) a protein transduction domain; and (ii) a MYC polypeptide sequence. In another aspect, the present disclosure provides a method for treating a chronic microbial infection in a subject in need thereof, wherein the method comprises administering an effective amount a PTD-MYC fusion protein comprising (i) a protein transduction domain; and (ii) a MYC polypeptide sequence. In some embodiments, the subject is identified as having altered expression of at least one or more immune cell markers associated with T cell exhaustion, or T cell impairment, compared to that observed in a healthy control. In some embodiments, the one or more modified immune cells are derived from immune cells isolated from the subject.

In some embodiments, the chronic microbial infection is a bacterial infection, a viral infection, a fungal infection, a protozoan infection, or parasitic infection. In some embodiments, the chronic microbial infection is chronic or latent form of a viral infection. In some embodiments, the chronic microbial infection is caused by a pathogen selected from the group consisting of Mycobacterium tuberculosis, Mycobacterium leprae, Mycobacterium bovis, Helicobacter pylori, Staphylococcus aureus, Salmonella Typhi, Treponema pallidum, Escherichia coli, Hemophilus influenza, Pseudomonas aeruginosa, Plasmodium falciparum, Plasmodium vivax, Plasmodium ovale, Plasmodium malariae, Human Immunodeficiency Virus (HIV), Herpesviruses, Herpes Simplex Virus (HSV), Hepatitis B Virus (HBV), Hepatitis C Virus (HCV), Measles Virus, Papovaviruses, Varicella-Zoster Virus, T-Cell Leukemia Viruses, Adenoviruses, Parvoviruses, Epstein-Barr Virus, Enterovirus, Mouse Hepatitis Virus (MHV), Cytomegalovirus (CMV), Papillomaviruses and Lymphocytic Choriomeningitis Virus (LCMV). In some embodiments, the chronic microbial infection is an antibiotic resistant or antiviral resistant infection (e.g., antibiotic resistant tuberculosis (TB), Methicillin-resistant Staphylococcus aureus (MRSA), Enterovirus 68, Nipah virus, Middle East respiratory syndrome (MERS)).

In another aspect, the present disclosure provides a method for treating an acute respiratory viral infection in a subject in need thereof, wherein the method comprises administering an effective amount of one or more modified immune cells (e.g., CD8⁺ T cells) to the subject, wherein the one or more modified immune cells comprise a PTD-MYC fusion protein comprising (i) a protein transduction domain; and (ii) a MYC polypeptide sequence. In another aspect, the present disclosure provides a method for treating an acute respiratory viral infection in a subject in need thereof, wherein the method comprises administering an effective amount a PTD-MYC fusion protein comprising (i) a protein transduction domain; and (ii) a MYC polypeptide sequence. In some embodiments, the subject is identified as having altered expression of at least one or more immune cell markers associated with T cell exhaustion, or T cell impairment, compared to that observed in a healthy control. In some embodiments, the one or more modified immune cells are derived from immune cells isolated from the subject.

In some embodiments, the subject has previously been administered one or more vaccines against the pathogen. In some embodiments, the subject exhibits T cell exhaustion, or T cell impairment, following vaccination against the pathogen. In some embodiments, the subject exhibits T cell exhaustion, or T cell impairment, during pathogenic infection, where the subject has been previously vaccinated against the pathogen.

In some embodiments, the subject having a chronic microbial infection, or acute respiratory viral infection, exhibits increased expression one or more immune cell markers, where increased expression of the immune cell marker is associated with T cell exhaustion, or T cell impairment, compared to that observed in a healthy control (e.g., a non-exhausted T cell). In some embodiments, the one or more immune cell markers that are increased is an immune checkpoint protein. In some embodiments, the one or more immune cell markers that are increased is selected from the group consisting of PD1, LAG3, CD160, 2B4 (CD244) and a combination of any two or more thereof. In some embodiments, the one or more immune cell markers that are increased is selected from the group consisting of PD-1, CTLA-4, LAG-3,CD160, 2B4, IL-7ra, IL-15ra, KLRG-1, TIM-3, Eomes and a combination of any two or more thereof. In some embodiments, the one or more immune cell markers that are increased is selected from the group consisting of CD244, PDCD1, CTLA4, GP49B, PTGER4, CD160, LAG3, IL7R, IL15RA, HAVCR2, PTGER2, CD7, TNFRSF9, GLYCOP, VCAM1, TNFSF6, ITM2A, MOX2, ITGAV, CD9,CCL3, CXCR4, CCL4, CCRL2, SMAD1, RGS16, GPR56, TANK, DUSP1, GPR65, PTPN13, PRKWNK, IL6ST, ITPR5, JAK3, MAP3K1, SH2D2A, SOCS3, ACTN1, ISG20, GIP2, ICSBP1, SERPINA, TCRG-V4, TCRB-V1, PBX3, EOMES, ATF1, AHR, EGR2, HFATC1, ZFP91, HIST1H2, BCL2, CASP3, GAS2, CKS2, CAR2, PLSCR1, SLC12A2, ART3, GPD2, NDUFA5, CYP4V3, ENTPD1, P2RX4, C76628, SFRS7, MTV43, C79248, PENK1, COCH, NR4A2, A43010, IER5, SEPTIN, RCN, WBP5, PBEF1, PPM1B, DDIT4, KDT1, TRIM47, HRB, RNF11, NRIP1, TUBB2, SPP1, KLK6, LY75, SCL29A1, BUB1, NDFIP1, SPRED2, CBX4, and a combination of any two or more thereof.

In some embodiments, the subject having a chronic microbial infection, or acute respiratory viral infection, exhibits decreased expression one or more immune cell markers, where decreased expression of the immune cell marker is associated with T cell exhaustion, or T cell impairment, compared to that observed in a healthy control (e.g., a non-exhausted T cell). In some embodiments, the one or more immune cell markers that is decreased is selected from the group consisting of IGSF10, SELPL, H2-Q8, FCGR2B, CD3D, ITGB2, ITGA1, ITGA4, KLRK1, SEMA4A, IL18R1, KLRD1, LY6C, KLRC1, EDG6, ITGB7, ITGAX, KLRG1, CKLFSF7, LSP1, CCR5, CCR2, H2-K1, H2-D1, H2-Q10, H2-Q7, SIAT10, LGALS10, RAC2, PIK3CD, ILK, MAP3K8, ARHGEF1, S100A6, STK38, LCK, ARHGAP1, S100A4, RASA3, JAK1, S100A1, HCPH, DGKA, PTK9L, IL18RAP, MAP1LC3, ACTB, IFNGR, TPP2, LYZS, CAPN2, CTSD, USP3, PRSS19, MUS, BZW1, RORA, KLF2, RUNX1, HOD, KLF3, SATB1, BNIP3L, ANXA2, ANXA6, ANXA1, VAMP3, SNX5, SNX10, CDC37, CDKN2D, PAK2, CDC34, HMGCS1, RPN2, NDUFB6, PDHA1, ARL6IP5, PSMD13, FXYD5, ARL2BP, UBE1X, APOBEC, KCTDI0, SSR3, UGCG, MGC68, GARS, SDHA, HSPA8, RRM2, ATPSL, PFKP, HBB-B1, HBB-A1, TCEB2, EIF3S8, RPS16, EIF2S1, SFPQ, RPLI0A, RPL36, EEF2, RPS7, PIGT, RPL27A, PPGB, TAGLN2, HINTI, LEFI, LSPI, TOBI, SDCBP, SEP15, MTVR2, GABARAPL2, PLD3, ETSI, DSTN, LBR, GOLPH2, WDRI, PLP2, NME2, KCNAB2, DIAPI, TXNDC5, SMFN, STARDI0, CLICI, TMSBI0, IAP, GM2A, ATP6V0B, DNAJDI, SUPT4H, PTPRC, CRIPI, EMPI, PLAC8, and a combination of any two or more thereof.

In any of the methods provided here, subjects for treatment with a MYC fusion protein provided herein or a modified immune cell comprising a MYC fusion protein provided herein can be a human or a non-human animal.

In another aspect, the present disclosure provides a method for treating a cancer in a subject in need thereof, wherein the method comprises administering an effective amount of one or more modified immune cells (e.g., CD8⁺ T cells) to the subject, wherein the one or more modified immune cells comprise a PTD-MYC fusion protein comprising (i) a protein transduction domain; and (ii) a MYC polypeptide sequence, where the subject is identified as having altered expression of at least one or more immune cell markers associated with T cell exhaustion, or T cell impairment, compared to that observed in a healthy control. In another aspect, the present disclosure provides a method for treating a cancer in a subject in need thereof, wherein the method comprises administering an effective amount a PTD-MYC fusion protein comprising (i) a protein transduction domain; and (ii) a MYC polypeptide sequence, where the subject is identified as having altered expression of at least one or more immune cell markers associated with T cell exhaustion, or T cell impairment, compared to that observed in a healthy control. In some embodiments, the one or more modified immune cells are derived from immune cells isolated from the subject. In some embodiments, the cancer is a metastatic cancer. In some embodiments, the cancer is a carcinoma, adenoma, adenocarcinoma, blastoma, sarcoma, or lymphoma. In some embodiments, the cancer is a basal cell carcinoma, biliary tract cancer, bladder cancer, breast cancer, cervical cancer, choriocarcinoma, CNS cancer, colon cancer, colorectal cancer, connective tissue cancer, cancer of the digestive system, endometrial cancer, esophageal cancer, eye cancer, gastric cancer, glial cell tumor, head and neck cancer, hepatoma, hepatic carcinoma, Hodgkin's lymphoma, Non-Hodgkin's lymphoma, intra-epithelial neoplasm, kidney cancer, larynx cancer, liver cancer, small-cell lung cancer, non-small cell lung cancer, melanoma, myeloma, neuroblastoma, oral cavity cancer, ovarian cancer, pancreatic cancer, prostate cancer, rectal cancer, renal cancer, cancer of the respiratory system, retinoblastoma, rhabdomyosarcoma, salivary gland carcinoma, squamous cell cancer, stomach cancer, testicular cancer, thyroid cancer, uterine cancer, cancer of the urinary system, or a vulvar cancer. In some embodiments, the immune cells are obtained from a donor subject having solid tumor where the subject is identified as having altered expression of at least one or more immune cell markers associated with T cell exhaustion, or T cell impairment, compared to that observed in a healthy control. In some embodiments, the solid tumor is a metastatic tumor.

### III. Methods of Obtaining and Preparing Immune Cells for Transfer

Immune cells for use in the methods provided herein can be obtained using any suitable method known in the art. In some embodiments, the immune cells are primary immune cells. In some embodiments, the immune cells are lymphocytes, such as T and B cells. In some embodiments, the immune cells are natural killer (NK) cells. In some embodiments, the immune cells are a mixture of lymphocytes and NK cells. In some embodiments, the immune cells are obtained from the peripheral blood of a donor subject. In some embodiments, the immune cells are peripheral blood mononuclear cells (PBMC). In some embodiments, the immune cells are obtained from the spleen or lymph nodes of a donor subject.

In some embodiments, the immune cells are modified following isolation from a donor. For example, in some embodiments, the immune cells are CART cells.

In some embodiments, the immune cells are obtained from a subject that has a chronic infection. In some embodiments, the chronic infection is a bacterial infection, a viral infection, a fungal infection, a protozoan infection, or parasitic infection.

In some embodiments, the immune cells are obtained from a subject that has a tumor. In some embodiments, the immune cells are T cells that have infiltrated a tumor (e.g., tumor infiltrating lymphocytes). In some embodiments, the T cells are removed during surgery of a tumor. For example, in some embodiments, the T cells are isolated after removal of tumor tissue by biopsy.

In some embodiments, the T cells are isolated from a sample containing a population of cells, such as a blood, lymph, spleen or tissue biopsy sample. T cells can be isolated from a population of cells by any means known in the art. In one embodiment, the methods comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells can include, but are not limited to, any one or more of mechanically dissociating (e.g., mincing) the tumor, enzymatically dissociating (e.g., digesting) the tumor, and/or aspiration (e.g., as with a needle).

The bulk population of T cells obtained from a tumor sample can comprise any suitable type of T cell. Preferably, the bulk population of T cells obtained from a tumor sample comprises tumor infiltrating lymphocytes (TILs).

The population of immune cells can be obtained from any mammal. Unless stated otherwise, as used herein, the term “mammal” refers to any mammal including, but not limited to, mammals of the order Logomorpha, such as rabbits; the order Camivora, including Felines (cats) and Canines (dogs); the order Artiodactyla, including Bovines (cows) and Swines (pigs); or of the order Perssodactyla, including Equines (horses). The mammals can be non-human primates, e.g., of the order Primates, Ceboids, or Simoids (monkeys) or of the order Anthropoids (humans and apes). In some embodiments, the mammal can be a mammal of the order Rodentia, such as mice and hamsters. Preferably, the mammal is a non-human primate or a human. An exemplary mammal is a human.

In some embodiments, the subject to receive the immune cells is also the donor of the immune cells (i.e., autologous ACT). In some embodiments, the subject to receive the immune cells is different than the donor of the tumor sample (i.e. allogenic ACT).

T cells can be obtained from a number of sources, including peripheral blood mononuclear cells, bone marrow, lymph node tissue, spleen tissue, and tumors. In certain embodiments, T cells can be obtained from a unit of blood collected from a subject using any number of techniques known to the skilled artisan, such as Ficoll separation. In one embodiment, cells from the circulating blood of an individual are obtained by apheresis or leukopheresis. The apheresis product typically contains lymphocytes, including T cells, monocytes, granulocytes, B cells, other nucleated white blood cells, red blood cells, and platelets. In one embodiment, the cells collected by apheresis can be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and can lack magnesium or can lack many if not all divalent cations. Initial activation steps in the absence of calcium lead to magnified activation. As those of ordinary skill in the art would readily appreciate, a washing step can be accomplished by methods known to those in the art, such as by using a semi-automated “flow-through” centrifuge (for example, the Cobe 2991 cell processor) according to the manufacturer's instructions. After washing, the cells can be resuspended in a variety of biocompatible buffers, such as, for example, Ca-free, Mg-free PBS. Alternatively, the undesirable components of the apheresis sample can be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one embodiment, T cells are isolated by incubation with anti-CD3/anti-CD28 (i.e., 3×28)—conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours. In one embodiment, the incubation time period is 24 hours. For isolation of T cells from patients with leukemia, use of longer incubation times, such as 24 hours, can increase cell yield. Longer incubation times can be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8⁺ T cells.

Enrichment of a T cell population by negative selection can be accomplished with a combination of antibodies directed to surface markers unique to the negatively selected cells. In one embodiment, the method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) can be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology can be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it can be desirable to significantly decrease the volume in which beads and cells are mixed together (i.e., increase the concentration of cells), to ensure maximum contact of cells and beads. For example, in one embodiment, a concentration of 2 billion cells/mL is used. In one embodiment, a concentration of 1 billion cells/mL is used. In a further embodiment, greater than 100 million cells/mL is used. In a further embodiment, a concentration of cells of 10, 15, 20, 25, 30, 35, 40, 45, or 50 million cells/mL is used. In yet another embodiment, a concentration of cells from 75, 80, 85, 90, 95, or 100 million cells/mL is used. In further embodiments, concentrations of 125 or 150 million cells/mL can be used. Using high concentrations can result in increased cell yield, cell activation, and cell expansion. Further, use of high cell concentrations allows more efficient capture of cells that can weakly express target antigens of interest, such as CD28− negative T cells, or from samples where there are many tumor cells present (e.g., leukemic blood, tumor tissue). Such populations of cells can have therapeutic value and would be desirable to obtain. For example, using high concentration of cells allows more efficient selection of CD8⁺ T cells that normally have weaker CD28 expression.

In a related embodiment, it can be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8⁺ T cells in dilute concentrations. In one embodiment, the concentration of cells used is 5×10⁶/mL. In other embodiments, the concentration used can be from about 1×10⁵/ml to 1×10⁶/mL, and any integer value in between. Thus, the concentration used may be from about 1×10⁵/mL, about 1.1×10⁵/mL, about I.2×10⁵/mL, about 1.3×10⁵/mL, about 1.4×10⁵/mL, about 1.5×10⁵/mL, about 1.6×10⁵/mL, about 1.7×10⁵/mL, about I.8×10⁵/mL, about 1.9×10⁵/mL, about 2×10⁵mL, about 2.2×10⁵/mL, about 2.4×10⁵/mL, about 2.6×10⁵/mL, about 2.8×10⁵/mL, about 3×10⁵/mL, about 3.2×10⁵/mL, about 3.4×10⁵/mL, about 3.6×10⁵/mL, about 3.8×10⁵/mL, about 4×10⁵/mL, about 4.2×10⁵/mL, about 4.4×10⁵/mL, about 4.6×10⁵/mL, about 4.8×10⁵/mL, about 5×10⁵/mL, about 5.5×10⁵/mL, about 6×10⁵/mL, about 6.5×10⁵/mL, about 7×10⁵/mL, about 7.5×10⁵/mL, about 8×10⁵/mL, about 8.5×10⁵/mL, about 9×10⁵/mL, about 9.5×10⁵/mL, about 1×10⁶/mL, or any integer value in between.

T cells can also be frozen. The freeze and subsequent thaw step can provide a more uniform product by removing granulocytes and to some extent monocytes in the cell population. After a washing step to remove plasma and platelets, the cells can be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 10 per minute and stored in the vapor phase of a liquid nitrogen storage tank. Other methods of controlled freezing can be used as well as uncontrolled freezing immediately at −20° C. or in liquid nitrogen.

In some embodiments, cells are directly labeled with an epitope-specific reagent for isolation and/or enrichment by flow cytometry followed by characterization of cell phenotypes. In some embodiments, immune cells are isolated by contacting the immune cell specific antibodies. Sorting of any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif).

In one embodiment, the method further comprises expanding the numbers of T cells in the enriched cell population. Such methods are described in U.S. Pat. No. 8,637,307 and is hereby incorporated by reference in its entirety. The T cells can be expanded before or after treatment of the cells with the PTD-MYC fusion polypeptide. The numbers of T cells can be increased at least about 3-fold (or 4-, 5-, 6-, 7-, 8-, or 9-fold), more preferably at least about IO-fold (or 20-, 30-, 40-, 50-, 60-, 70-, 80-, or 90-fold), more preferably at least about I 00-fold, more preferably at least about 1,000-fold, or most preferably at least about 100,000-fold. The numbers of T cells can be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells can be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal can be used in soluble form. Ligands can be attached to the surface of a cell, to an Engineered Multivalent Signaling Platform (EMSP), or immobilized on a surface. In a one embodiment both primary and secondary agents are co-immobilized on a surface, for example a bead or a cell. In one embodiment, the molecule providing the primary activation signal can be a CD3 ligand, and the co-stimulatory molecule can be a CD28 ligand or 4-1BB ligand. In some embodiments, the cells are expanded by stimulation with one or more antigens, such as a melanoma tumor antigen or antigens derived from the patient's tumor.

In some embodiments, the isolated immune cells are immediately treated with the PTD-MYC fusion polypeptide following isolation. In other embodiments, the isolated immune cells are stored in a suitable buffer and frozen prior to treatment with the PTD-MYC fusion polypeptide. In some embodiments, the isolated immune cells are immediately treated with the PTD-MYC fusion polypeptide following isolation and the treated cells are stored in a suitable buffer and frozen until needed for administration to the patient.

In certain embodiments, the isolated immune cells (e.g., a mixed population immune cells or isolated types, such as CD8⁺ T cells) are contacted with a composition containing a PTD-MYC fusion polypeptide for a period of time sufficient to be taken up by the cells. In some embodiments, the immune cells are contacted with a composition containing a PTD-MYC fusion polypeptide for less than about 24 hours, less than about 23 hours, less than about 22 hours, less than about 21 hours, less than about 20 hours, less than about 19 hours, less than about 18 hours, less than about 17 hours, less than about 16 hours, less than about 15 hours, less than about 14 hours, less than about 13 hours, less than about 12 hours, less than about 11 hours, less than about 10 hours, less than about 9 hours, less than about 8 hours, less than about 7 hours, less than about 6 hours, less than about 5 hours, less than about 4 hours, less than about 3 hours, less than about 2 hours, or less than about 1 hour.

In certain embodiments, the immune cells are contacted with a composition containing a PTD-MYC fusion polypeptide for less than about 55 minutes, less than about 50 minutes, less than about 45 minutes, less than about 40 minutes, less than about 35 minutes, less than about 30 minutes, less than about 29 minutes, less than about 28 minutes, less than about 27 minutes, less than about 26 minutes, less than about 25 minutes, less than about 24 minutes, less than about 23 minutes, less than about 22 minutes, less than about 21 minutes, less than about 20 minutes, less than about 19 minutes, less than about 18 minutes, less than about 17 minutes, less than about 16 minutes, less than about 15 minutes, less than about 14 minutes, less than about 13 minutes, less than about 12 minutes, less than about 11 minutes, or less than about 10 minutes. In certain embodiments, the immune cells are contacted with a composition containing a PTD-MYC fusion polypeptide for about 1 hour.

In certain embodiments, the immune cells are contacted with a composition containing a PTD-MYC fusion polypeptide for 24 hours or longer. In certain embodiments, the immune cells are contacted with a composition containing a PTD-MYC fusion polypeptide for less than about 12 days, less than about 11 days, less than about 10 days, less than about 9 days, less than about 8 days, less than about 7 days, less than about 6 days, less than about 5 days, less than about 4 days, less than about 2 days, or less than about 1 day.

In certain embodiments that may be combined with any of the preceding embodiments, the cells are contacted with a PTD-MYC fusion polypeptide at a concentration of 0.5 μg/ml to 500 μg/ml. 0.5 μg/ml, at least 0.6 μg/ml, at least 0.7 μg/ml, at least 0.8 μg/ml, at least 0.9 μg/ml, at least 1 μg/ml, at least 2 μg/ml, at least 3 μg/ml, at least 4 μg/ml, at least 5 μg/ml, at least 6 μg/ml, at least 7 μg/ml, at least 8 μg/ml, at least 9 μg/ml, at least 10 μg/ml, at least 15 μg/ml, at least 20 μg/ml, at least 25 μg/ml, at least 30 μg/ml, at least 35 μg/ml, at least 40 μg/ml, at least 45 μg/ml, at least 50 μg/ml, at least 55 μg/ml, at least 60 μg/ml, at least 65 μg/ml, at least 70 μg/ml, at least 75 μg/ml, at least 80 μg/ml, at least 85 μg/ml, at least 90 μg/ml, at least 95 μg/ml, at least 100 μg/ml, at least 110 μg/ml, at least 120 μg/ml, at least 130 μg/ml, at least 140 μg/ml, at least 150 μg/ml, at least 160 μg/ml, at least 170 μg/ml, at least 180 μg/ml, at least 190 μg/ml, at least 200 μg/ml, at least 220 μg/ml, at least 240 μg/ml, at least 260 μg/ml, at least 280 μg/ml, at least 300 μg/ml, at least 320 μg/ml, at least 340 μg/ml, at least 360 μg/ml, at least 380 μg/ml, at least 400 μg/ml, at least 420 μg/ml, at least 440 μg/ml, at least 460 μg/ml, at least 480 μg/ml, at least 500 μg/ml.

In some embodiments, the immune cells that are contacted with a composition containing a PTD-MYC fusion polypeptide are T cells with genetically modified antigen receptors, including chimeric antigen receptor (CAR)-T cells. Various strategies can, for example, be employed to genetically modify T cells by altering the specificity of the T cell receptor (TCR), for example, by introducing new TCR a and B chains with selected peptide specificity (see, e.g., U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379). Chimeric antigen receptors (CARs) can be used in order to generate immunoresponsive cells, such as T cells, specific for selected targets, such as malignant cells, with a wide variety of receptor chimera constructs having been described (see, e.g., U.S. Pat. Nos. 5,843,728; 5,851,828; 5,912,170; 6,004,811; 6,284,240; 6,392,013; 6,410,014; 6,753,162; 8,211,422; and, PCT Publication WO9215322). Methods for the preparation of CART cells are known in the art and can be used in combination with the methods provided herein to generate modified CART cells comprising a Myc fusion polypeptide (e.g., PTD) as described herein. In some embodiments, PTD-MYC fusion polypeptide can improve the expansion of the CART cells prior to administration to the subject.

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

In some embodiments, the T cells expressing a desired CAR are selected through co-culture with γ-irradiated activating and propagating cells (AaPC), which co-express the antigen and co-stimulatory molecules. In some embodiments, the engineered CAR T-cells are expanded, for example by co-culture on AaPC in presence of soluble factors, such as IL-2 and IL-21. This expansion can for example be carried out so as to provide memory CAR+ T cells. In this way, CAR T cells can be provided that have specific cytotoxic activity against antigen-bearing cells (optionally in conjunction with production of desired chemokines such as interferon-γ).

In some embodiments, the CART-cells are contacted with a PTD-MYC fusion polypeptide provided herein in vitro to generation a modified CART cells for the treatment of a disease or condition associated with T cell exhaustion or T cell impairment (e.g., a chronic viral infection, cancer, or acute respiratory viral infection). The modified CART cells can be administered according any suitable method, including the methods for administration of the PTD-MYC fusion polypeptide-modified immune cells as described above.

### IV. MYC Fusion Proteins

In some embodiments, the PTD-MYC fusion polypeptide comprises a protein transduction domain (PTD), a MYC polypeptide that promotes one or more of cell survival or proliferation, and optionally a protein tag domain, e.g., one or more amino acid sequences that facilitate purification of the fusion protein. In some embodiments, a cell contacted with MYC polypeptide exhibits increased survival time (e.g., as compared to an identical or similar cell of the same type that was not contacted with MYC), and/or increased proliferation (e.g., as compared to an identical or similar cell of the same type that was not contacted with MYC).

In some embodiments, the MYC fusion protein comprises (a) a protein transduction domain; and (b) a MYC polypeptide sequence. In some embodiments, the MYC fusion protein is a polypeptide of Formula (I):
protein transduction domain-MYC polypeptide sequence.

In some embodiments, a MYC fusion protein disclosed herein comprises (a) a protein transduction domain; (b) a MYC polypeptide sequence; and (c) one or more molecules that link the protein transduction domain and the MYC polypeptide sequence. In some embodiments, the MYC fusion protein is a polypeptide of Formula (II):
protein transduction domain-X-MYC polypeptide sequence,
wherein -X- is a molecule that links the protein transduction domain and the MYC polypeptide sequence. In some embodiments, -X- is at least one amino acid.

In some embodiments, a MYC fusion protein disclosed herein comprises (a) a protein transduction domain; (b) a MYC polypeptide sequence; (c) at least two protein tags; and (d) optionally linker(s). In some embodiments, the MYC fusion protein is a polypeptide of Formula (III-VI):
protein transduction domain-X-MYC polypeptide sequence-X-protein tag 1-X-protein tag 2 (Formula (III)), or
protein transduction domain-MYC polypeptide sequence-X-protein tag 1-X-protein tag 2 (Formula (IV)), or
protein transduction domain-MYC polypeptide sequence-protein tag 1-X-protein tag 2 (Formula (V)), or
protein transduction domain-MYC polypeptide sequence-protein tag I-protein tag 2 (Formula (VI)),
wherein -X- is a linker. In some embodiments, -X- is one or more amino acids.

In some embodiments, a MYC fusion protein disclosed herein comprises (a) a protein transduction domain; (b) a MYC polypeptide sequence; (c) a 6-histidine tag; (d) a VS epitope tag: and (e) optionally linker(s). In some embodiments, the MYC fusion protein is a polypeptide of Formula (VII-XIV):
protein transduction domain-X-MYC polypeptide sequence-X-6-histidine tag-X-V5 epitope tag (Formula (VII)), or
protein transduction domain-MYC polypeptide sequence-X-6-histidine tag-X-V5 epitope tag (Formula (VIII)), or
protein transduction domain-MYC polypeptide sequence-6-histidine tag-X-V5 epitope tag (Formula (IX)), or
protein transduction domain-MYC polypeptide sequence-6-histidine tag-VS epitope tag (Formula (X)),
protein transduction domain-X-MYC polypeptide sequence-X-V5 epitope tag-X-6-histidine tag (Formula (XI)), or
protein transduction domain-MYC polypeptide sequence-X-V5 epitope tag-X-6-histidine tag (Formula (XII)), or
protein transduction domain-MYC polypeptide sequence-VS epitope tag-X-6-histidine tag (Formula (XIII)), or
protein transduction domain-MYC polypeptide sequence-VS epitope tag-6-histidine tag (Formula (XIV)),
wherein -X-i s a linker. In some embodiments, -X- is one or more amino acids.

As noted above, in some embodiments, the MYC fusion protein comprises one or more linker sequences. The linker sequences can be employed to link the protein transduction domain, MYC polypeptide sequence, VS epitope tag and/or 6-histidine tag of the fusion protein. In some embodiments, the linker comprises one or more amino acids. In some embodiments, the amino acid sequence of the linker comprises KGELNSKLE. In some embodiments, the linker comprises the amino acid sequence of RTG.

sProtein Transduction Domain (PTD)

In some embodiments, the MYC fusion protein includes a protein transduction domain. Peptide transport provides an alternative for delivery of small molecules, proteins, or nucleic acids across the cell membrane to an intracellular compartment of a cell. One non-limiting example and well-characterized protein transduction domain (PTD) is a TAT-derived peptide. Frankel et al. (see, e.g., U.S. Pat. Nos. 5,804,604, 5,747,641, 5,674,980, 5,670,617, and 5,652,122) demonstrated transport of a cargo protein (β-galactosidase or horseradish peroxidase) into a cell by conjugating a peptide containing amino acids 48-57 of TAT to the cargo protein. In some embodiments, TAT comprises an amino acid sequence of MRKKRRQRRR (SEQ ID NO: 7).

Another non-limiting example of a PTD is penetratin. Penetratin can transport hydrophilic macromolecules across the cell membrane (Derossi et al., Trends Cell Biol., 8:84-87 (1998) incorporated herein by reference in its entirety). Penetratin is a 16 amino acid peptide that corresponds to amino acids 43-58 of the homeodomain of Antennapedia, a Drosophila transcription factor which is internalized by cells in culture.

Yet another non-limiting example of a PTD is VP22. VP22, a tegument protein from Herpes simplex virus type 1 (HSV-1), has the ability to transport proteins and nucleic acids across a cell membrane (Elliot et al., Cell 88:223-233, 1997, incorporated herein by reference in its entirety). Residues 267-300 of VP22 are necessary but cannot be sufficient for transport. Because the region responsible for transport function has not been identified, the entire VP22 protein is commonly used to transport cargo proteins and nucleic acids across the cell membrane (Schwarze et al., Trends Pharmacol Sci, 21:45-48, 2000).

In some embodiments, the PTD-MYC fusion polypeptide includes a protein transduction domain. By way of example, but not by way of limitation, in some embodiments, the protein transduction domain comprises the protein transduction domain of one or more of TAT, penetratin, VP22, vpr, EPTD, R9, R15, VP16, and Antennapedia. In some embodiments, the protein transduction domain comprises the protein transduction domain of one or more of TAT, penetratin, VP22, vpr, and EPTD. In some embodiments, the protein transduction domain comprises the protein transduction domain of at least one of TAT, penetratin, VP22, vpr, EPTD, R9, R15, VP16, and Antennapedia. In some embodiments, the protein transduction domain comprises a synthetic protein transduction domain (e.g., polyarginine or PTD-5). In particular embodiments, the protein transduction domain comprises a TAT protein transduction domain. In some embodiments, the protein transduction domain is covalently linked to the MYC polypeptide. In some embodiments, the protein transduction domain is linked to the MYC polypeptide via a peptide bond. In some embodiments, the protein transduction domain is linked to the MYC polypeptide via a linker sequence. In some embodiments, the linker comprises a short amino acid sequence. By way of example, but not by way of limitation, in some embodiments, the linker sequence may be 1, 2, 3, 4, 5, 6, 7, 8, 9, or 10 amino acids in length.

The PTD-MYC fusion protein of the present technology can be arranged in any desired order. For example, in some embodiments, the MYC fusion protein can be arranged in order of a) the protein transduction domain linked in frame to the MYC polypeptide, b) the MYC polypeptide linked in frame to the VS domain, and c) the VS domain linked in frame to the 6-histidine epitope tag. In some embodiments, the MYC fusion protein has an order of components of a) the MYC polypeptide linked in frame to the protein transduction domain, b) the protein transduction domain linked in frame to the VS domain, and c) the VS domain linked in frame to the 6-histidine epitope tag. In some embodiments, additional amino acid sequences can be included between each of the sequences. In some embodiments, additional amino acids can be included at the start and/or end of the polypeptide sequences.

In some embodiments, the protein transduction domain is a TAT protein transduction domain. In some embodiments, the protein transduction domain is TAT^{[48-57]}. In some embodiments, the protein transduction domain is TAT^{[57-48]}.

### Protein Tag Domains

In some embodiments, the MYC fusion protein comprises a protein tag domain that comprises one or more amino acid sequences that facilitate purification of the fusion protein. In some embodiments, the protein tag domain comprises one or more of a polyhistidine tag, and an epitope tag. By way of example, but not by way of limitation, exemplary tags include one or more of a VS, a histidine-tag (e.g., a 6-histidine tag), HA (hemagglutinin) tags, FLAG tag, CBP (calmodulin binding peptide), CYD (covalent yet dissociable NorpD peptide), Strepll, or HPC (heavy chain of protein C). In some embodiments, the protein tag domain comprises about 10 to about 20 amino acids in length. In some embodiments, the protein tag domain comprises 2 amino acids to 40 amino acids in length, for example 6-20 amino acids in length. In some embodiments, the protein tag domain comprises 2 amino acids, 3 amino acids, 4 amino acids, 5 amino acids, 6 amino acids, 7 amino acids, 8 amino acids, 9 amino acids, 10 amino acids, 11 amino acids, 12 amino acids, 13 amino acids, 14 amino acids, 15 amino acids, 16 amino acids, 17 amino acids, 18 amino acids, 19 amino acids, 20 amino acids, 21 amino acids, 22 amino acids, 23 amino acids, 24 amino acids, 25 amino acids, 26 amino acids, 27 amino acids, 28 amino acids, 29 amino acids, 30 amino acids, 31 amino acids, 32 amino acids, 33 amino acids, 34 amino acids, 35 amino acids, 36 amino acids, 37 amino acids, 38 amino acids, 39 amino acids, or 40 amino acids. In some embodiments, two of the above listed tags (for example, VS and the 6-histidine-tag) are used together to form the protein tag domain.

In some embodiments, the histidine tag is a 6-histidine tag. In some embodiments, the histidine tag comprises the sequence HHHHHH (SEQ ID NO: 8). In some embodiments, the MYC fusion protein disclosed herein comprises a VS epitope tag. In some embodiments, the VS tag comprises the amino acid sequence of: GKPIPNPLLGLDST (SEQ ID NO:9). In some embodiments, the VS tag comprises the amino acid sequence ofIPNPLLGLD (SEQ ID NO: 10).

The protein tags can be added to the fusion protein disclosed herein by any suitable method. By way of example, but not by way of limitation, in some embodiments, a TAT-MYC polypeptide sequence is cloned into an expression vector encoding one or more protein tags, e.g., a polyHis-tag and/or a VS tag. In some embodiments, a polyhistidine tag and/or a VS tag is added by PCR (i.e., the PCR primers comprise a polyhistidine sequence and/or VS sequence).

### Construction of PTD-MYC Fusion Polypeptides

PTD-MYC fusion polypeptides (e.g., TAT-MYC fusion polypeptide) disclosed herein can be constructed by methods well known in the art. By way of example, but not by way of limitation, a nucleotide sequence encoding a TAT-MYC fusion polypeptide can be generated by PCR. In some embodiments, a forward primer for a human MYC sequence comprises an in frame N-terminal 9-amino-acid sequence of the TAT protein transduction domain (e.g., RKKRRQRRR). In some embodiments, a reverse primer for a human MYC sequence is designed to remove the stop codon. In some embodiments, the PCR product is cloned into any suitable expression vector. In some embodiments, the expression vector comprises a polyhistidine tag and a VS tag.

In some embodiments, a MYC fusion protein disclosed herein comprises (a) TAT, and (b) c-MYC. In some embodiments, a MYC fusion protein disclosed herein comprises (a) TAT[48-57J, and (b) c-MYC. In some embodiments, a MYC fusion protein disclosed herein comprises (a) TAT^{[57-48]}, and (b) c-MYC.

In some embodiments, a MYC fusion protein disclosed herein comprises (a) TAT, (b) c-MYC, (c) linker(s), (d) VS tag, and (e) 6-histidine tag. In some embodiments, a MYC fusion protein disclosed herein comprises (a) TAT^{[48-57]}, (b) c-MYC, (c) linker(s), (d) VS tag, and (e) 6-histidine tag. In some embodiments, a MYC fusion protein disclosed herein comprises (a) TAT^{[57-48]}, (b) c-MYC, (c) linker(s), (d) VS tag, and (e) 6-histidine tag.

In some embodiments, the PTD-MYC fusion polypeptide comprises SEQ ID NO: 1; in some embodiments, the PTD-MYC fusion polypeptide is SEQ ID NO: 1.

(SEQ ID NO: 1)
MRKKRRQRRRPLNVSFTNRNYDLDYDSVQPYFYCDEEENFYQQQ
QQSELQPPAPSEDIWKKFELLPTPPLSPSRRSGLCSPSYVAVTP
FSLRGDNDGGGGSFSTADQLEMVTELLGGDMVNQSFICDPDDET
FIKNIIIQDCMWSGFSAAAKLVSEKLASYQAARKDSGSPNPARG
HSVCSTSSLYLQDLSAAASECIDPSVVFPYPLNDSSSPKSCASQ
DSSAFSPSSDSLLSSTESSPQGSPEPLVLREETPPTTSSDSEEE
QEDEEEIDVVSVEKRQAPGKRSESGSPSAGGHSKPPHSPLVLKR
CHVSTHQHNYAAPPSTRKDYPAAKRVKLDSVRVLRQISNNRKCT
SPRSSDTEENVKRRTHNVLERQRRNELKRSFFALRDQIPELENN
EKAPKVVILKKATAYILSVQAEEQKLISEEDLLRKRREQLKEIK
LEQLRKGELNSKLEGKPIPNPLLGLDSTRTGHHHHHH.

The fusion protein can be modified during or after synthesis to include one or more functional groups. By way of example but not by way of limitation, the protein can be modified to include one or more of an acetyl, phosphate, acetate, amide, alkyl, and/or methyl group. This list is not intended to be exhaustive, and is exemplary only. In some embodiments, the protein includes at least one acetyl group.

A PTD-MYC fusion polypeptide can be generated by any suitable method known the art, e.g. by recombinant protein expression in a cell, such as a bacterial cell, an insect cell, or mammalian cell. In some embodiments, a PTD-MYC fusion polypeptide is recombinantly produced by microbial fermentation. In some embodiments microbial fermentation is performed in a fermentation volume of from about 1 to about 10,000 liters, for example, a fermentation volume of about 10 to about 1,000 liters. The fermentation can utilize any suitable microbial host cell and culture medium. In exemplary embodiments, E. coli is utilized as the microbial host cell. In alternative embodiments, other microorganisms can be used, e.g., S. cerevisiae, P. pastoris, Lactobacilli, Bacilli and Aspergilli. In an exemplary embodiment the microbial host cell is BL-21 Star1M E. coli strain (Invitrogen). In an exemplary embodiment the microbial host cell is BLR DE3 E. coli. strain.

In some embodiments the host cells are modified to provide tRNAs for rare codons, which are employed to overcome host microbial cell codon bias to improve translation of the expressed proteins. In exemplary embodiments, the host cells (e.g., E. coli) transformed with a plasmid, such as pRARE (CamR), which express tRNAs for AGG, AGA, AUA, CUA, CCC, GGA codons. Additional, suitable plasmids or constructs for providing tRNAs for particular codons are known in the art and can be employed in the methods provided.

Integrative or self-replicative vectors can be used for the purpose of introducing the PTD-MYC fusion polypeptide expression cassette into a host cell of choice. In an expression cassette, the coding sequence for the PTD-MYC fusion polypeptide is operably linked to promoter, such as an inducible promoter. Inducible promoters are promoters that initiate increased levels of transcription from DNA under their control in response to some change in culture conditions, e.g., the presence or absence of a nutrient or a change in temperature. In some embodiments, the nucleic acid encoding the PTD-MYC fusion polypeptide is codon optimized for bacterial expression.

Exemplary promoters that are recognized by a variety of potential host cells are well known. These promoters can be operably linked to PTD-MYC fusion polypeptide-encoding DNA by removing the promoter from the source DNA, if present, by restriction enzyme digestion and inserting the isolated promoter sequence into the vector. Promoters suitable for use with microbial hosts include, but are not limited to, the β-lactamase and lactose promoter systems (Chang et al. (1978) Nature, 275:617-624; Goeddel et al. (1979) Nature, 281:544), alkaline phosphatase, a tryptophan (trp) promoter system (Goeddel (1980) Nucleic Acids Res. 8:4057; EP 36,776), and hybrid promoters such as the tac promoter (deBoer et al. (1983) Proc. Natl. Acad. Sci. USA 80:21-25). Any promoter for suitable for expression by the selected host cell can be used. Nucleotide sequences for suitable are published, thereby enabling a skilled worker operably to ligate them to DNA encoding PTD-MYC fusion polypeptide (see, e.g., Siebenlist et al. (1980) Cell 20:269) using linkers or adaptors to supply any required restriction sites. In exemplary embodiments, promoters for use in bacterial systems can contain a Shine-Dalgarno (S.D.) sequence operably linked to the coding sequence. In some embodiments, the inducible promoter is the lacZ promoter, which is induced with Isopropyl β-D-1-thiogalactopyranoside (IPTG), as is well-known in the art. Promoters and expression cassettes can also be synthesized de nova using well known techniques for synthesizing DNA sequences of interest. In an exemplary embodiment, the expression vector for expression of the PTD-MYC fusion polypeptides herein is pET101/D-Topo (Invitrogen).

For expression of the PTD-MYC fusion polypeptides, the microbial host containing the expression vector encoding the PTD-MYC fusion polypeptide is typically grown to high density in a fermentation reactor. In some embodiments, the reactor has controlled feeds for glucose. In some embodiments, a fermenter inoculum is first cultured in medium supplemented with antibiotics (e.g., overnight culture). The fermenter inoculum is then used to inoculate the fermenter culture for expression of the protein. At an OD600 of at least about 15, usually at least about 20, at least 25, at least about 30 or higher, of the fermenter culture, expression of the recombinant protein is induced. In exemplary embodiments, where the inducible promoter is the lacZ promoter, IPTG is added to the fermentation medium to induce expression of the PTD-MYC fusion polypeptide. Generally, the IPTG is added to the fermenter culture at an OD600 which represents logarithmic growth phase.

In certain embodiments of the methods provided, induced protein expression is maintained for around about 2 to around about 5 hours post induction, and can be from around about 2 to around about 3 hours post-induction. Longer periods of induction may be undesirable due to degradation of the recombinant protein. The temperature of the reaction mixture during induction is preferably from about 28° C. to about 37° C., usually from about 30° C. to about 37° C. In particular embodiments, induction is at about 37° C.

The PTD-MYC fusion polypeptide is typically expressed as cytosolic inclusion bodies in microbial cells. To harvest inclusion bodies, a cell pellet is collected by centrifugation of the fermentation culture following induction, frozen at −70° C. or below, thawed and resuspended in disruption buffer. The cells are lysed by conventional methods, e.g., sonication, homogenization, etc. The lysate is then resuspended in solubilization buffer, usually in the presence of urea at a concentration effective to solubilize proteins, e.g., from around about SM, 6M, 7M, 8M, 9M or greater. Resuspension may require mechanically breaking apart the pellet and stirring to achieve homogeneity. In some embodiments, the cell pellet is directly resuspended in urea buffer and mixed until homogenous. In some embodiments, the resuspension/solubilization buffer is 8M Urea, 50 mM Phosphate pH 7.5 and the suspension is passed through a homogenizer.

In some embodiments, the homogenized suspension is sulfonylated. For example, in some embodiments, the homogenized suspension is adjusted to include 200 mM Sodium Sulfite and 10 mM Sodium Tetrathionate. The solution is then mixed at room temperature until homogeneous. The mixed lysate is then mixed for an additional period of time to complete the sulfonylation (e.g., at 2-8° C. for ≥12 hours). The sulfonylated lysate was then centrifuged for an hour. The supernatant containing the sulfonylated PTD-MYC fusion polypeptides is then collected by centrifugation and the cell pellet discarded. The supernatant is then passed through a filter, e.g., 0.22 μm membrane filter to clarify the lysate.

The solubilized protein is then purified. Purification methods may include affinity chromatography, reversed-phase chromatography, gel exclusion chromatography, and the like. In some embodiments, affinity chromatography is used. For example, the protein is provided with an epitope tag or 6-histidine-tag for convenient purification. In the present methods, exemplary PTD-MYC fusion polypeptides comprise a 6-histidine-tag for purification using Ni affinity chromatography using Ni-resin.

[In exemplary embodiments, the Ni-resin column is equilibrated in a buffer containing urea. In some embodiments, the equilibration buffer is 6M Urea, 50 mM Phosphate, 500 mM NaCl, and 10% Glycerol solution. The sulfonylated and clarified supernatant comprising the PTD-MYC fusion polypeptide is then loaded onto the Ni-resin column. The column is then washed with a wash buffer, e.g., 6M Urea, 50 mM Phosphate, 10% Glycerol, 500 mM NaCl, pH 7.5. The column was then washed with sequential wash buffers with decreasing salt concentration. For example, exemplary subsequent washed can include 6M Urea, 50 mM Phosphate, 10% Glycerol, and 2M NaCl, pH 7.5, followed another wash of 6M Urea, 50 mM Phosphate, 10% Glycerol, 50 mM NaCl, and 30 mM Imidazole, pH 7.5.

Following sequential application of the wash buffers the PTD-MYC fusion polypeptide is eluted from the column by addition of elution buffer, e.g., 6M Urea, 50 mM Phosphate, 10% Glycerol, and 50 mM NaCl, pH 7.5 with a gradient from 100 to 300 mM Imidazole, and collecting fractions. The protein containing fractions to be pooled are then filtered through a 0.22 um membrane. Assessment of protein yield can be measured using any suitable method, e.g., spectrophotometry at UV wavelength 280.

In some embodiments, one or more additional purification methods can be employed to further purify the isolated PTD-MYC fusion polypeptides. In exemplary embodiments, the pooled fractions from the Ni-Sepharose chromatography step are further purified by anion exchange chromatography using a Q-Sepharose resin. In some embodiments, the pool is prepared for loading onto the Q-Sepharose column by diluting the samples to the conductivity of the Q Sepharose buffer (17.52 +/−1 mS/cm) with the second wash buffer (e.g., 6M Urea, 50 mM Phosphate, 10% Glycerol, 2M NaCl, pH 7.5) from the Ni Sepharose chromatography step. The diluted pool is then loaded onto the Q-Sepharose column, followed by two chase steps using a chase buffer (e.g., 6M Urea, 50 mM Phosphate, 300 mM NaCl, and 10% Glycerol), with further sequential applications of the chase buffer until the UV trace reaches baseline, indicating that the protein has eluted from the column.

### V. Administration of the PTD-MYC Fusion Proteins and PTD-MYC Modified Immune Cells to a Subject

As provided previously, the present disclosure provides a method for treating an T cell exhaustion, or T cell impairment, in a subject in need thereof, wherein the method comprises administering an effective amount of a MYC fusion protein comprising (i) a protein transduction domain; and (ii) a MYC polypeptide sequence or one or more immune cells comprising the MYC fusion protein. In some embodiments, the methods comprise administering an effective amount of modified immune cell comprising the MYC fusion protein.

Any method known to those in the art for contacting a cell, organ or tissue with one or more PTD-MYC fusion polypeptides disclosed herein may be employed. Suitable methods include in vitro, ex vivo, or in vivo methods. In vivo methods typically include the administration of one or more PTD-MYC fusion polypeptides to a mammal, suitably a human. In vitro or ex vivo methods typically include the administration of one or more modified immune cells that have been treated with a PTD-MYC fusion polypeptides to a mammal, suitably a human. The one or more PTD-MYC fusion polypeptides or PTD-MYC modified immune cells described herein are administered to the subject in effective amounts (i.e., amounts that have desired therapeutic effect). The dose and dosage regimen will depend upon the degree of the disease state of the subject, the characteristics of the particular PTD-MYC fusion polypeptide used, e.g., its therapeutic index, and the subject's history.

The effective amount may be determined during pre-clinical trials and clinical trials by methods familiar to physicians and clinicians. An effective amount of one or more PTD-MYC fusion polypeptides or PTD-MYC modified immune cells useful in the methods disclosed herein may be administered to a mammal in need thereof by any of a number of well-known methods for administering pharmaceutical compounds and cells. The PTD-MYC fusion polypeptides or PTD-MYC modified immune cells may be administered systemically or locally.

For the administration of the PTD-MYC modified immune cell compositions, any suitable method for administration of cells to a subject may be employed. For example, suitable methods for adoptive cell therapy typically involve systemic infusion (e.g., intravenous or intraperitoneal infusion) of the modified immune cells together with a pharmaceutically acceptable carrier, diluent or excipient. In some embodiments, the PTD-MYC modified immune cells are administered locally, for example, intratumorally or at the site of infection.

In some embodiments, the one or more PTD-MYC fusion polypeptides described herein can be incorporated into pharmaceutical compositions for administration, singly or in combination, to a subject for the treatment or prevention of a microbial infection. In some embodiments, the one or more PTD-MYC fusion polypeptides or modified immune cells described herein can be incorporated into pharmaceutical compositions for administration, singly or in combination, to a subject for the treatment or prevention of immune cell exhaustion. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more PTD-MYC fusion polypeptides disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of PTD-MYC fusion polypeptides described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8) 915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly a-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more PTD-MYC fusion polypeptides disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more PTD-MYC fusion polypeptides concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more PTD-MYC fusion polypeptides may be defined as a concentration of inhibitor at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### VI. Combination Therapies

In some embodiments, the PTD-MYC fusion polypeptides or the PTD-MYC modified immune cells are administered with an additional therapeutic agent. Suitable therapeutic agents for combination therapy can be selected based on the condition or disease to be treated. In some embodiments, additional therapeutic agent is administered prior to, simultaneously with, intermittently with, or following treatment with the PTD-MYC fusion polypeptides or the PTD-MYC modified immune cells. In some embodiments, the additional therapeutic agent is an immunomodulator, such as an interleukin (e.g. IL-2, IL-7, IL-12), a cytokine, a chemokine, or and immunomodulatory drug. In some embodiments, the additional therapeutic agent is an anticancer agent (e.g., chemotherapy, radiation therapy, oncolytic agent, immunotherapy, monoclonal antibodies, anti-cancer nucleic acids or proteins, anti-cancer viruses or microorganisms, and any combinations thereof), an antibacterial agent or an antiviral agent.

### VII. Kits

Kits according to this embodiment can comprise a carrier means, such as a box, carton, tube, having in close confinement therein one or more containers, such as vials, tubes, ampoules, bottles, syringes, or bags. The kits can also comprise associated instructions for using the PTD-MYC fusion polypeptides and/or PTD-MYC fusion polypeptide-modified immune cells of the present technology. In some embodiments, the kit comprises an effective amount of an adoptive cell therapy, such as MYC-fusion polypeptide-modified immune cells. In some embodiments, the kit comprises one for more reagents for the detection of the administered MYC-fusion polypeptides and/or MYC-fusion polypeptide-modified immune cells.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Isolated Immune Cells Express Decreased Levels of Immune Checkpoint Receptors PD-1 and CTLA-4 After Treatment With TAT-MYC

A whole blood sample was isolated from a donor subject and mixed with the blood anticoagulant, ethylenediaminetetraacetic acid (EDTA). After allowing the cells to incubate at least 24 hours at 37° C., 5% CO₂, the whole blood sample is then separated into peripheral blood mononuclear cells (PBMCs; immune cells), which include lymphocytes (e.g., T cells, B cells, NK cells) and monocytes, and waste (i.e., red blood cells, platelets, plasma, etc.) using a density-gradient solution (DGS) on a SEPAX-100 cell processing system (Biosafe America Inc., Houston, TX). The PBMCs were washed twice during the cell separation process with a 2.5% (w/v) HSA (Human Serum Albumin) solution in saline. Following the wash step, the immune cells were resuspended in the 2.5% (w/v) HSA solution at a concentration of 1×106 cells/ml to provide a cell suspension.

Following the cell separation process, samples of the immune cells were treated with DPBS (negative control) or TAT-MYC fusion protein (25 μg/mL for 10⁶ cells) and incubated at room temperature for 1 hour. The treated immune cells (called TBX-3400) were then re-washed on the SEPAX-100, and excess TAT-MYC was washed off of the cells with the 2.5% (w/v) HSA solution. Following the final wash step, the TBX-3400 were resuspended in in the 2.5% (w/v) HSA solution at a concentration of 1×106 cells/ml.

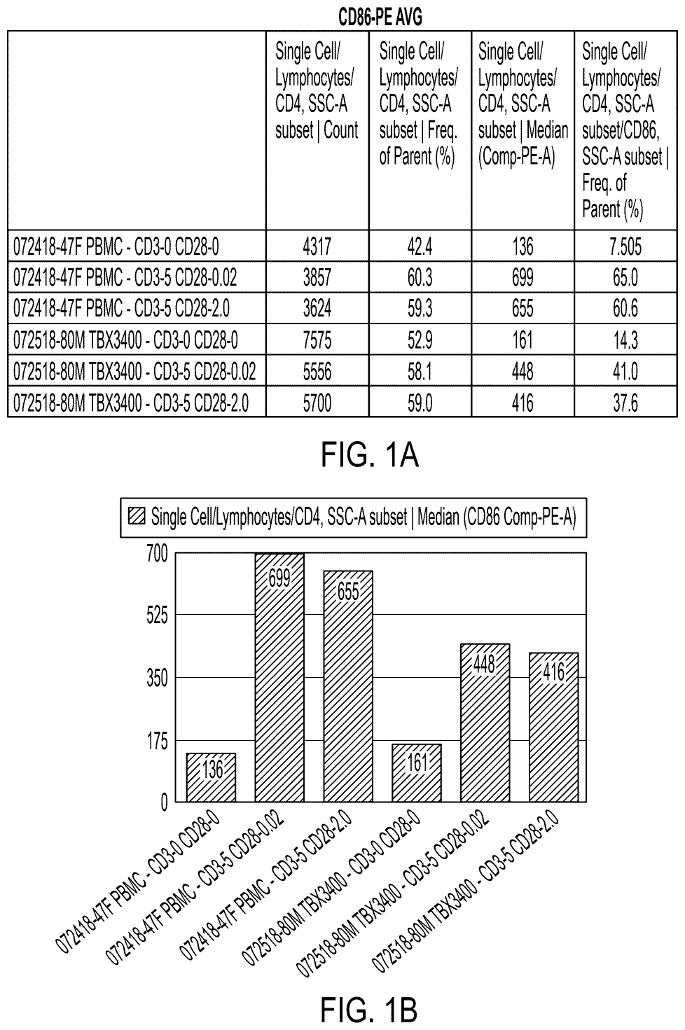

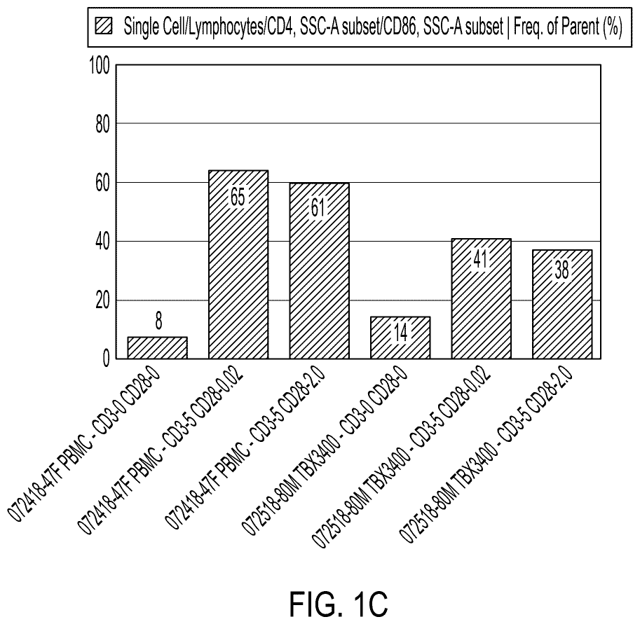

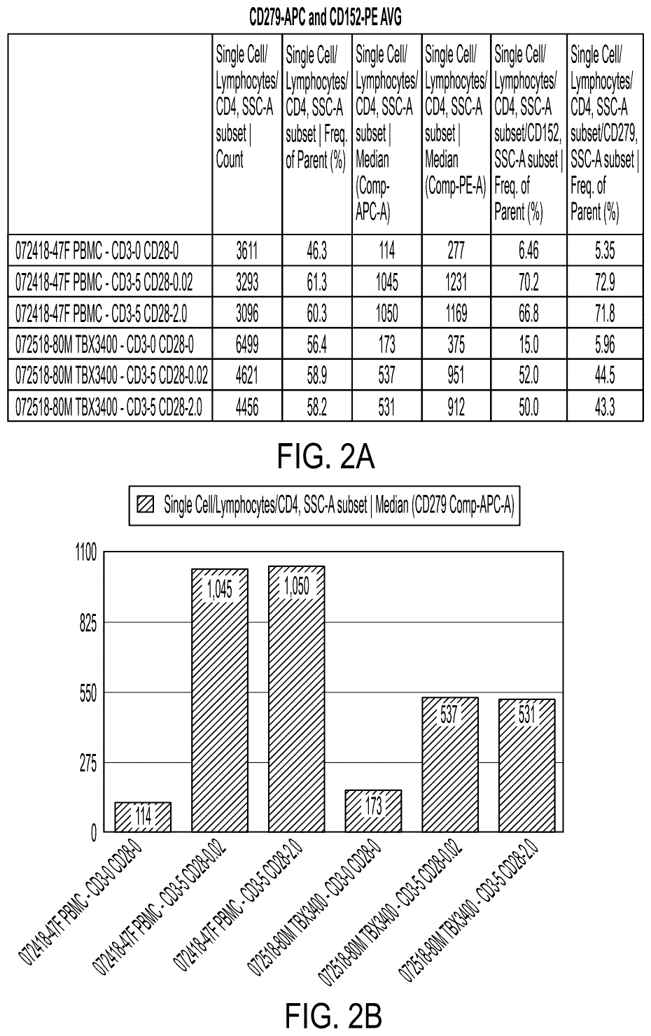

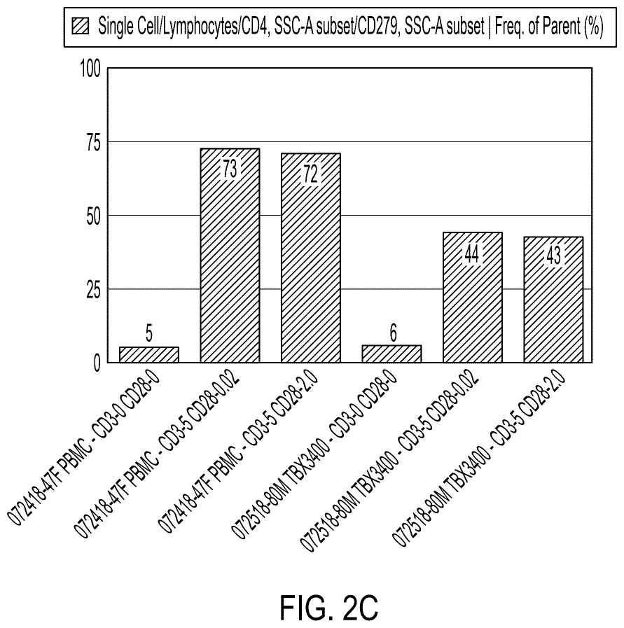

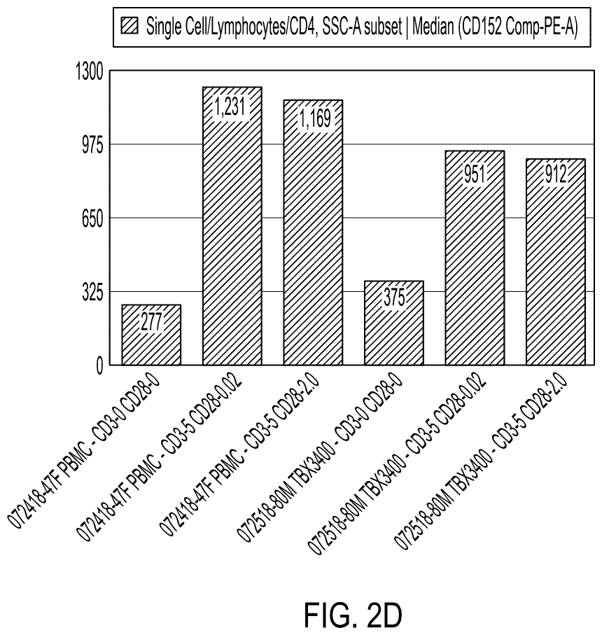

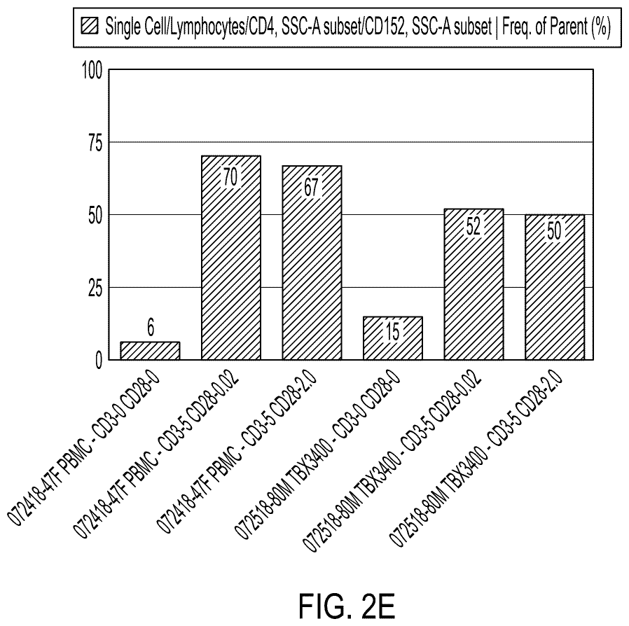

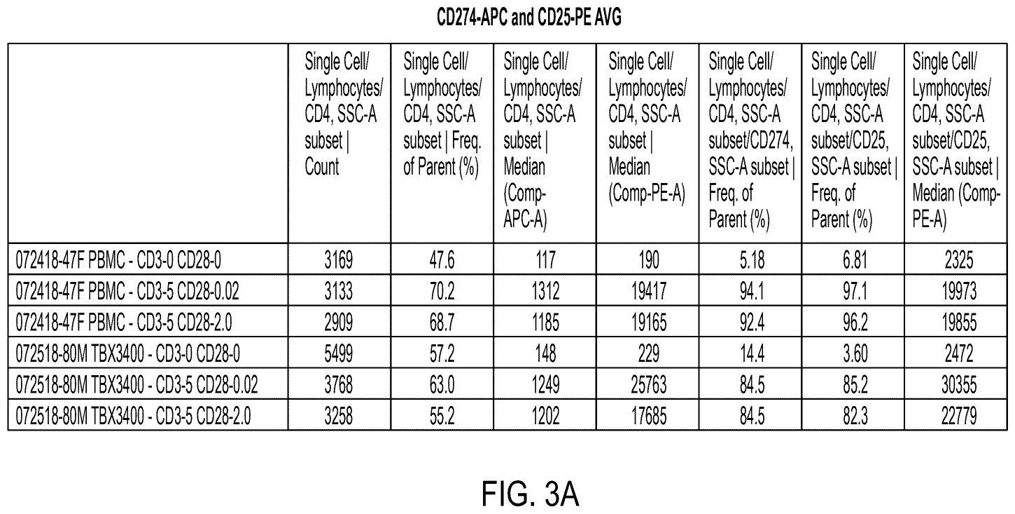

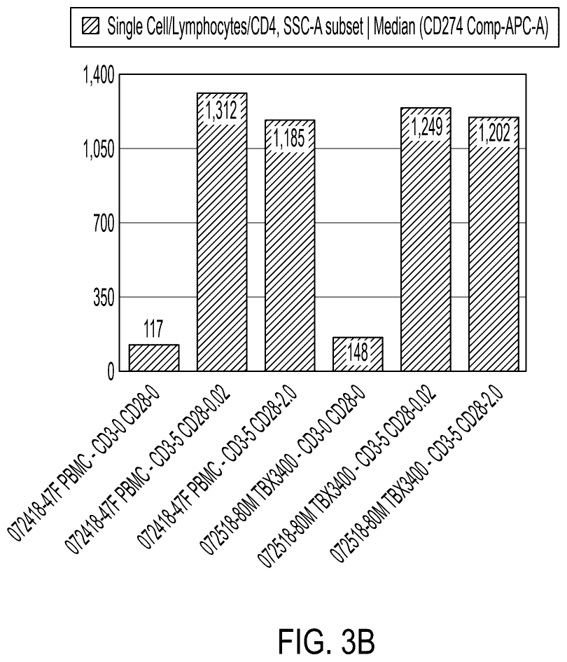

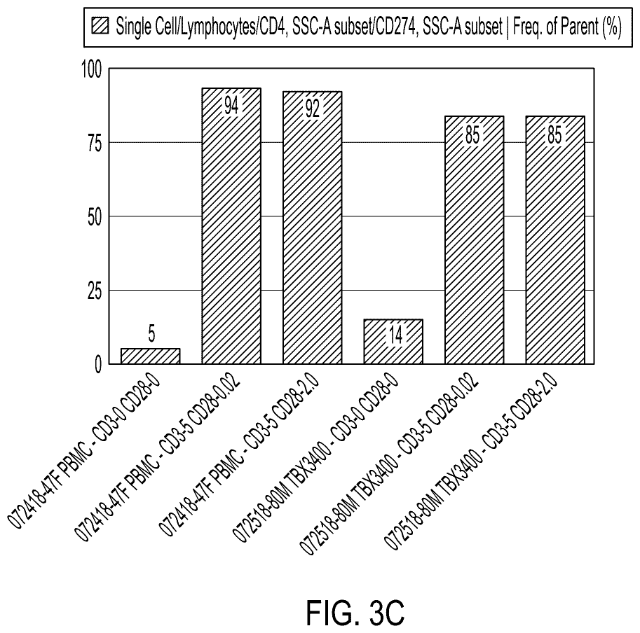

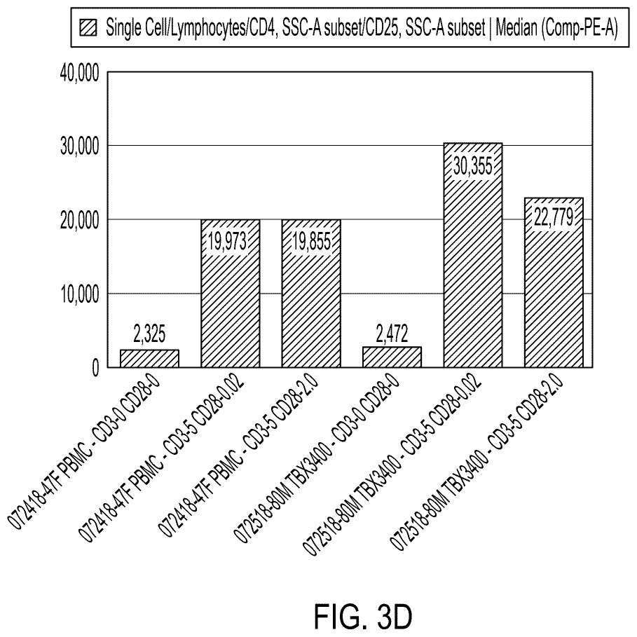

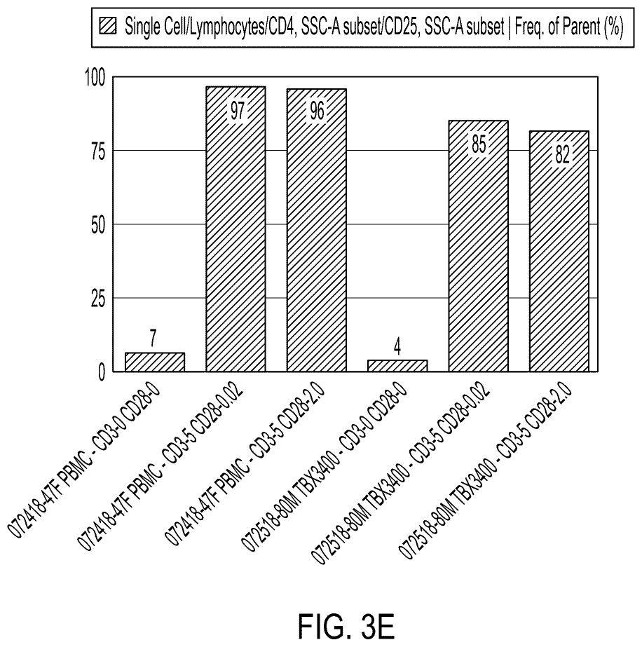

A 24-well plate was coated with a solution of an anti-CD3e antibody (500 μL, 5 μg/mL; BD Biosciences) in sterile DPBS. For control wells, only 500 μL of DPBS was added. The plates were allowed to incubated overnight at 4° C. prior to removing the solutions. Each well was then washed twice with 2 mL of sterile DPBS. Cells were then resuspended in complete RPMI medium (cRPMI) at a concentration of 2×106 cells/mL, and subsequently washed with 1 mL of DPBS. Next, 1.0 mL of the cell suspension was added to each well according to the plate layout. Next, a solution of an anti-CD28 antibody (100 μL, 200 μg/mL; BD Biosciences) was prepared in cRPMI and serially diluted 10-fold to make two stock solutions of the CD28 antibody in cRPMI (20 μg/mL, and 2 μg/mL). Next, 10 μL of the appropriate CD28 antibody solution or DPBS (controls or singly activated cells) was added to the designated wells. Assay plates were then incubated at 37° C., 5% CO₂ for 48 or 72 hours, followed by staining with the appropriate antibodies (anti-human CD25-PE, anti-human CD274-APC, anti-human CD279-APC, anti-human CD152-PE, and anti-human CD86-PE; BD Biosciences) for visualization of the magnitude of cell surface expression of immune checkpoint proteins by FACS analysis (FIGS. 1-3). FIGS. 2A-2E illustrate that the TBX-3400 demonstrates decreased expression levels of PD-1 and CTLA-4, compared to control immune cells, as determined by flow cytometry after cell activation.

Accordingly, these results demonstrate that the PTD-MYC fusion proteins disclosed herein are useful in methods for decreasing immune checkpoint proteins expressed on T cells and ameliorating and/or reversing T cell exhaustion in a subject in need thereof.

### Example 2: Isolated Immune Cells Express Decreased Expression of Immune Cell Markers of Dysfunction After Treatment With TAT-MYC

Peripheral blood mononuclear cells (PBMCs) are prepared as described above. Following the cell separation process, samples of the immune cells are treated with DPBS (negative control) or TAT-MYC fusion protein (25 μg/mL for 106 cells) and incubated at room temperature for 1 hour. The treated immune cells (called TBX-3400) are then re-washed on the SEPAX-100, and excess TAT-MYC is washed off of the cells with the 2.5% (w/v) HSA solution. Following the final wash step, the TBX-3400 are resuspended in in the 2.5% (w/v) HSA solution at a concentration of 1×106 cells/ml.

A 24-well plate is coated with a solution of an anti-CD3e antibody (500 μL, 5 μg/mL; BD Biosciences) in sterile DPBS. For control wells, only 500 μL of DPBS was added. The plates were allowed to incubate overnight at 4° C. prior to removing the solutions. Each well is then washed twice with 2 mL of sterile DPBS. Cells are then resuspended in complete RPMI medium (cRPMI) at a concentration of 2×106 cells/mL, and subsequently washed with 1 mL of DPBS. Next, 1.0 mL of the cell suspension is added to each well according to the plate layout. Next, a solution of an anti-CD28 antibody (100 μL, 200 μg/mL; BD Biosciences) is prepared in cRPMI and serially diluted IO-fold to make two stock solutions of the CD28 antibody in cRPMI (20 μg/mL, and 2 μg/mL). Next, 10 μL of the appropriate CD28 antibody solution or DPBS (controls or singly activated cells) is added to the designated wells. Assay plates are then incubated at 37° C., 5% CO2 for 48 or 72 hours, followed by staining with the antibodies to LAG3, CD160, 2B4, IL-7ra, IL-15ra, KLRG-1, TIM-3, and Eomes for visualization of the magnitude of cell surface expression of these immune cell markers of immune cell dysfunction proteins by FACS analysis. It is expected that TAT-MYC treated cells (TBX-3400) exhibit decreased expression levels of LAG3, CD160, 2B4, IL-7ra, IL-15ra, KLRG-1, TIM-3, and Eomes, compared to control immune cells, as determined by flow cytometry after cell activation.

Accordingly, these results will demonstrate that the PTD-MYC fusion proteins disclosed herein are useful in methods for decreasing cell surface expression of immune cell markers of immune cell dysfunction expressed on T cells and ameliorating and/or reversing T cell exhaustion, or T cell impairment in a subject in need thereof.

### Example 3: Mice Treated With TAT-MYC Exhibit Decreased Expression of Immune Cell Markers of Dysfunction After Chronic Viral Infection

In this example, mice will be treated with the PTD-MYC fusion protein of the present technology to determine whether the T cells in mice can be rescued from T cell exhaustion characterized by the increased expression of immune cell markers of T cell dysfunction after a chronic viral infection. Briefly, 2×10⁵ plaque-forming units (PFU) of mouse hepatitis virus (MHV), cytomegalovirus (CMV), hepatitis C virus (HEPC) or lymphocytic choriomeningitis virus (LCMV), are injected i.v. into the mice to initiate chronic infection. At a pre-determined time point post-infection (e.g. 1-10 weeks post infection), the mice are separated into two groups and are treated with 50 ug/kg PTD-MYC fusion protein (e.g., daily, weekly or biweekly) or not treated (NT; control group) via s.c. tail vein injection.

Immune cells isolated from mouse hepatitis virus (MHV), cytomegalovirus (CMV), or lymphocytic choriomeningitis virus (LCMV) infected mice and control mice are then stained with the appropriate fluorochrome-labeled antibodies for visualization of the magnitude of cell surface expression of immune checkpoint proteins by FACS analysis. It is anticipated that mice which exhibit a chronic microbial infection (NT; control group) will demonstrate elevated levels of immune cell markers of dysfunction including, but not limited to, PD-I, CTLA-4, and KLRG-1. Further, it is expected that mice treated with the PTD-MYC fusion protein of the present technology will demonstrated decreased levels of immune cell markers of dysfunction.

Accordingly, it is expected that these results will demonstrate that the PTD-MYC fusion proteins disclosed herein are useful in methods for treating chronic viral infection, characterized by an increase in immune checkpoint proteins, a decrease cell surface expression of cell markers of immune cell dysfunction, and ameliorating and/or reversing immune cell exhaustion in a subject in need thereof. Further, it is expected that treatment with a MYC fusion protein results in an increase in cytokine production (e.g., IL-2, TNF), Granzyme B, and/or IFN gamma expression in the subject compared to no treatment.

### Example 4: Mice Treated With TAT-MYC-Treated Immune Cells for Treatment of Chronic Viral Infection

In this example, mice having a chronic viral infection are treated with immune cells isolated from the lymph node or spleen and treated with the PTD-MYC fusion protein (e.g., TBX-3400). Briefly, 2×10⁵ plaque-forming units (PFU) of mouse hepatitis virus (MHV), cytomegalovirus (CMV), hepatitis C virus (HEPC) or lymphocytic choriomeningitis virus (LCMV), are injected i.v. into the mice to initiate chronic infection. At a pre-determined time points post-infection (e.g. 1-10 weeks post infection), the mice are separated into groups. Immune cells isolated from the lymph node or spleen that have been treated with the PTD-MYC fusion protein (e.g. TBX-3400) are administered to the mice intravenously via tail vein injection either during the initial wave of infection (to prevent chronic disease) or after the viral spike has passed and T cell function begins to decline (to treat chronic disease). The mice are assayed for expression of one or more immune checkpoint proteins and/or markers of immune cell exhaustion.

It is expected that mice treated with TBX-3400 will exhibit decreased viral titers and/or a decrease in one or more symptoms of chronic viral infection compared to control mice that are not treated with TBX-3400. Accordingly, it is expected that the treated mice will exhibit an increase in one or more immune checkpoint proteins, a decrease cell surface expression of cell markers of immune cell dysfunction, and amelioration and/or reversal of immune cell exhaustion compared to control mice that are not treated with TBX-3400. Further, it is expected that treatment with TBX-3400 results in an increase in cytokine production (e.g., IL-2, TNF), Granzyme B, and/or IFN gamma expression in the subject compared to no treatment.

### Example 5: Mice Treated With TAT-MYC Exhibit Decreased Expression of Immune Cell Markers of Dysfunction After Microbial Vaccination

In this example, mice will be treated with the PTD-MYC fusion protein of the present technology to determine whether the T cells in mice can be rescued from the increased expression of immune cell markers of dysfunction after microbial vaccination. Briefly, BALB\c mice are vaccinated with M bovis bacille Calmette-Guerin (BCG) either via intranaseal (i.n.) vaccination or subcutaneous (s.c.) vaccination. The i.n. vaccination is carried out by applying a total of 30 mL of the BCG vaccine suspension containing 1x106 CFU drop-wise to the external nares using a micropipette and allowing the mouse to inhale the suspension into the lungs naturally. For s.c. vaccination, 50 mL of a BCG suspension is injected into the both, the right and left flanks, using a needle to deliver a total of 1×10⁶ CFU. The vaccine diluent-administered or nai:ve mice serve as controls. BCG-vaccinated and control mice are separated into two groups and are treated with 50 ug/kg PTD-MYC fusion protein or not injected (NT-BCG-vaccinated; control group) via s.c. tail vein injection. At a pre-determined time point post-infection (e.g. 1-10 weeks post infection), the mice are challenged by the airway i.n. route to deliver 5×104 CFU of Mtb Erdman.

Lung or spleen cells harvested from BCG-vaccinated and control mice are then stained with the appropriate fluorochrome-labeled antibodies for visualization of cell surface expression levels of immune checkpoint proteins by FACS analysis. It is anticipated that NT-BCG-vaccinated mice will have elevated levels of immune cell markers of dysfunction including but not limited to, PD-I, CTLA-4, and KLRG-1. Further, it is expected that mice treated with the PTD-MYC fusion protein of the present technology will demonstrated decreased levels of immune cell markers of dysfunction.

Accordingly, it is anticipated that these results will demonstrate that the PTD-MYC fusion proteins disclosed herein are useful in methods for maintaining T-cell function in response to a microbial vaccine by increasing the expression of immune checkpoint proteins, decreasing cell surface expression of cell markers of immune cell dysfunction, and ameliorating and/or reversing T cell exhaustion in a subject in need thereof. Further, it is expected that treatment with a MYC fusion protein results in an increase in cytokine production (e.g., IL-2, TNF), Granzyme B, and/or IFN gamma expression in the subject compared to no treatment.

### Example 6: Mice Treated With TAT-MYC-Treated Immune Cells for Treatment of Chronic Microbial Infection

In this example, mice having a chronic microbial infection are treated with immune cells isolated from the lymph node or spleen and treated with the PTD-MYC fusion protein (e.g., TBX-3400). Briefly, mice are infected with a microbial pathogen (e.g., M bovis bacille Calmette-Guerin (BCG)) to initiate chronic infection. At a pre-determined time points post-infection (e.g. 1-10 weeks post infection), the mice are separated into groups. Immune cells isolated from the lymph node or spleen that have been treated with the PTD-MYC fusion protein (e.g. TBX-3400) are administered to the mice intravenously via tail vein injection either during the initial wave of infection (to prevent chronic disease) or after the initial spike of infection has passed and T cell function begins to decline (to treat chronic disease). The mice are assayed for expression of one or more immune checkpoint proteins and/or markers of immune cell exhaustion.

It is expected that mice treated with TBX-3400 will exhibit decreased CFU and/or a decrease in one or more symptoms of chronic microbial infection compared to control mice that are not treated with TBX-3400. Accordingly, it is expected that the treated mice will exhibit an increase in one or more immune checkpoint proteins, a decrease cell surface expression of cell markers of immune cell dysfunction, and amelioration and/or reversal of immune cell exhaustion compared to control mice that are not treated with TBX-3400. Further, it is expected that treatment with TBX-3400 results in an increase in cytokine production (e.g., IL-2, TNF), Granzyme B, and/or IFN gamma expression in the subject compared to no treatment.

### Example 7: Mice Treated With TAT-MYC Exhibit Decreased Expression of Immune Cell Markers of Dysfunction After Acute Respiratory Viral Infection

In this example, mice will be treated with the PTD-MYC fusion protein of the present technology to determine whether the T cells in mice can be rescued from T cell impairment characterized by the increased expression of immune cell markers of T cell dysfunction after a chronic viral infection. Briefly, about 2×10⁵ plaque-forming units (PFU) of a respiratory virus (e.g., influenza virus, respiratory syncytial virus (RSV), pneumonia virus, respiratory vaccinia virus, parainfluenza virus, respiratory adenoviruses, severe acute respiratory syndrome corona virus (SARS-CoV), Middle East respiratory syndrome corona virus (MERS-CoV), or human metapneumovirus (HMPV)) are administered the mice to initiate viral infection. At a pre-determined time point post-infection (e.g. 1-10 weeks post infection), the mice are separated into two groups and are treated with 50 ug/kg PTD-MYC fusion protein (e.g., daily, weekly or biweekly) or not treated (NT; control group) via s.c. tail vein injection.

Immune cells isolated from virus-infected mice and control mice are then stained with the appropriate fluorochrome-labeled antibodies for visualization of the magnitude of cell surface expression of immune checkpoint proteins by FACS analysis. It is anticipated that mice which exhibit T-cell impairment during an acute respiratory viral infection (NT; control group) will demonstrate elevated levels of immune cell markers of dysfunction including, but not limited to, PD-I, CTLA-4, and KLRG-1. Further, it is expected that mice treated with the PTD-MYC fusion protein of the present technology will demonstrated decreased levels of immune cell markers of dysfunction.

Accordingly, it is anticipated that these results will demonstrate that the PTD-MYC fusion proteins disclosed herein are useful in methods for treating chronic viral infection, characterized by an increase in immune checkpoint proteins, a decrease cell surface expression of cell markers of immune cell dysfunction, and amelioration and/or reversal of immune cell exhaustion/impairment in a subject in need thereof. Further, it is expected that treatment with TBX-3400 results in an increase in cytokine production (e.g., IL-2, TNF), Granzyme B, and/or IFN gamma expression in the subject compared to no treatment.

### Example 8: Mice Treated With TAT-MYC Exhibit Decreased Expression of Immune Cell Markers of Dysfunction After Acute Respiratory Viral Infection

In this example, mice will be treated with the PTD-MYC fusion protein of the present technology to determine whether the T cells in mice can be rescued from T cell impairment characterized by the increased expression of immune cell markers of T cell dysfunction after a chronic viral infection. Briefly, about 2×105 plaque-forming units (PFU) of a respiratory virus (e.g., influenza virus, respiratory syncytial virus (RSV), pneumonia virus, respiratory vaccinia virus, parainfluenza virus, respiratory adenoviruses, severe acute respiratory syndrome corona virus (SARS-CoV), Middle East respiratory syndrome corona virus (MERS-CoV), or human metapneumovirus (HMPV)) are administered the mice to initiate infection. At a pre-determined time point post-infection (e.g. 1-10 weeks post infection), the mice are separated into groups. Immune cells isolated from the lymph node or spleen that have been treated with the PTD-MYC fusion protein (e.g. TBX-3400) are administered to the mice intravenously via tail vein injection. The mice are assayed for expression of one or more immune checkpoint proteins and/or markers of immune cell exhaustion.

It is expected that mice treated with TBX-3400 will exhibit decreased viral titers and/or a decrease in one or more symptoms of viral infection compared to control mice that are not treated with TBX-3400. Accordingly, it is expected that the treated mice will exhibit an increase in one or more immune checkpoint proteins, a decrease cell surface expression of cell markers of immune cell dysfunction, and amelioration and/or reversal of immune cell exhaustion compared to control mice that are not treated with TBX-3400. Further, it is expected that treatment with TBX-3400 results in an increase in cytokine production (e.g., IL-2, TNF), Granzyme B, and/or IFN gamma expression in the subject compared to no treatment.

### Example 9: Isolated Immune Cells From Patients With Relapsed Refractory Melanoma Express Decreased Levels of Immune Checkpoint Receptors PD-1 and CTLA-4 After Treatment With TAT-MYC

This Example demonstrates that Tat-MYC fusion protein treatment of peripheral blood mononuclear cells (PBMCs) isolated from patients with relapsed refractory melanoma results in decreased surface expression levels of CD279 (PD-1) and CD152 (CTLA-4) on cells treated with Tat-MYC fusion protein.

Human PBMCs are obtained from peripheral blood by Ficoll-Hypaque gradient centrifugation. The cells are washed twice in 0.9% saline supplemented with 5% human serum albumin (HSA). The cells are then incubated for I hour with 50 μg/mL of Tat-MYC protein in 0.9% saline and 5% HSA, at room temperature. After the incubation, the cells are washed in protein in 0.9% saline and 5% HSA three times and resuspended in that buffer for further analysis and clinical use.

Some of the cells are centrifuged and resuspended in lymphocyte growth medium. This medium consists of RPMI medium supplemented with 10% human serum, penicillin/streptomycin, L-glutamine, non-essential amino acids and 213-mercaptoethanol. The cells are activated using plate bound anti-human CD3 antibody and soluble anti-CD28 antibodies for 4-6 days. Once the cells have been activated, they are collected, washed, and stained for flow cytometric analysis. The FACS analysis relies on the use of monoclonal antibodies directly conjugated to fluorochromes.

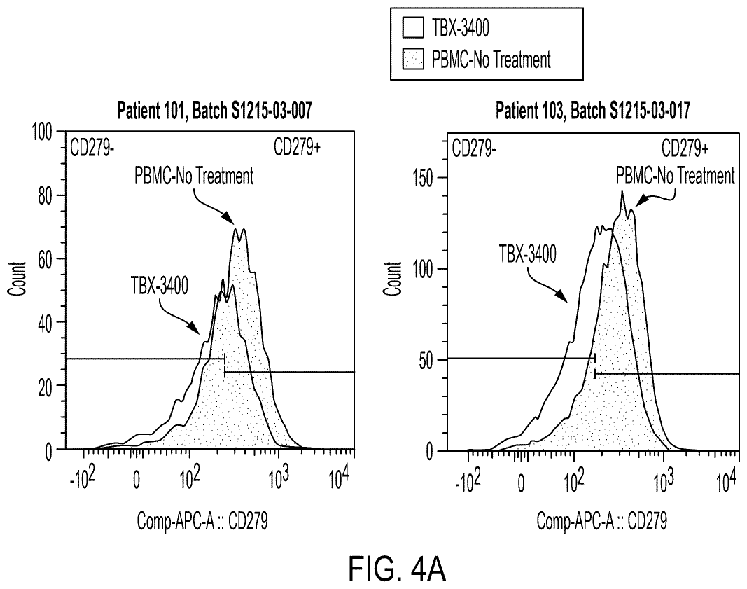

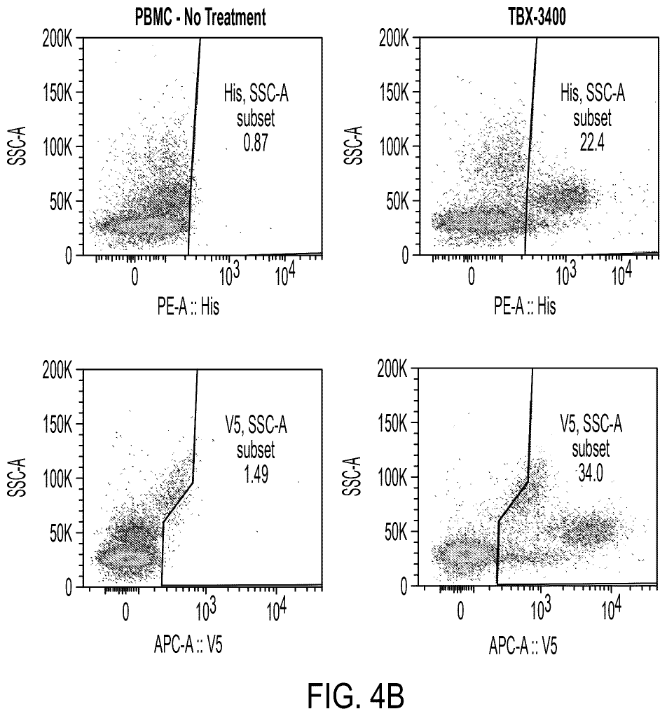

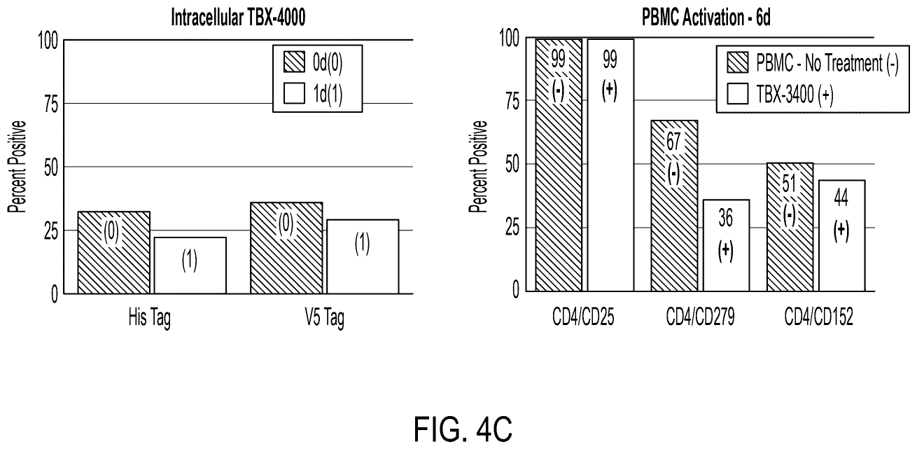

FIG. 4A shows the results from an analysis of blood collected from two patients (101 and 103) enrolled in a clinical study for relapsed refractory melanoma. As described above, the collected blood was processed to remove red blood cells and purify PBMCs. A sample was taken of the PBMCs before and after a one-hour treatment with the Tat-MYC protein. The cells were washed and cultured with CD3 and CD28 antibodies for T cell activation. The activated T cells were labeled with fluorescent antibodies to CD279 (PD-I) and analyzed by flow cytometry. As shown in FIG. 4A, in both patients, there was a measurable downregulation of CD279 (PD-I) in the Tat-MYC treated cells (TBX-3400) compared with the untreated cells (PBMC-No treatment).

Accordingly, treatment with Tat-MYC fusion protein reduces CD279 (PD-I) expression in immune cells isolated from melanoma patients.

FIG. 4B shows the results of a study of PBMCs purified from a patient enrolled in the relapsed refractory melanoma trial. The patient's PBMCs were either left untreated (left panels) or treated with Tat-MYC (TBX-4000) for one hour (right panels). The diagrams show the intracellular staining results for the His or VS tags. As shown in FIB. 4B, more than 20% of the cells treated with protein (TBX-3400) have internalized the protein.

FIG. 4C shows the results of a study on PBMCs treated with Tat-MYC (TBX-4000) for one hour in a closed system. Samples were analyzed by flow cytometry for intracellular levels of the protein by using fluorescent antibodies that bind to two different tags encoded in Tat-MYC (His Tag and VS Tag). As shown in FIG. 4C (left panel), over 25% of the cells stained positive for the protein on day zero (Od) and similar levels were detected the following day (Id). Tat-MYC treated cells (TBX-3400) and untreated cells (PBMC-No Treatment) isolated from the patient were activated with antibodies to CD3 and CD28, and the expression levels of the T cell activation marker, CD25, and the checkpoint markers, CD279 (PD-I) and CD152 (CTLA-4), were analyzed by flow cytometry. As shown in FIG. 4C (right panel), Tat-MYC treated cells had similar levels of CD25 yet lower levels of both checkpoint markers when compared with untreated cells.

Accordingly, these results demonstrate that the PTD-MYC fusion proteins disclosed herein are useful in methods for decreasing immune checkpoint proteins expressed on T cells and ameliorating and/or reversing T cell exhaustion and are useful in methods for treating T cell exhaustion in a subject in need thereof

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

1. A method for treating T cell exhaustion in a subject that has cancer, comprising administering an effective amount of one or more modified immune cells to the subject, wherein the immune cells are lymphocytes, natural killer cells, myeloid cells, or combinations thereof, and wherein the one or more modified immune cells comprises a MYC fusion protein comprising (i) a protein transduction domain; and (ii) a MYC polypeptide sequence.

2. The method of claim 1, wherein the subject is identified as having altered expression of at least one immune cell marker of T cell exhaustion compared to expression of the at least one immune cell marker in a healthy control.

3. The method of claim 2, wherein the immune cell marker is an immune checkpoint protein.

4. (canceled)

5. (canceled)

6. (canceled)

7. (canceled)

8. (canceled)

9. The method of claim 1, wherein the one or more modified immune cells are derived from immune cells isolated from the subject.

10. The method of claim 9, wherein the immune cells isolated from the subject are obtained from the peripheral blood, the lymph node, spleen, or tumor of the subject.

11. (canceled)

12. The method of claim 1, wherein the one or more modified immune cells are prepared by contacting the immune cells in vitro with the MYC fusion protein.

13.-14. (canceled)

15. The method of claim 1, wherein the protein transduction domain is a TAT protein transduction domain sequence.

16. The method of claim 1, wherein the MYC fusion protein comprises SEQ ID NO: 1.

17.-18. (canceled)

19. The method of claim 2, wherein the altered expression comprises an increase in the cell surface expression of one or more immune cell receptors.

20.-89. (canceled)

90. The method of claim 1, wherein the cancer is a carcinoma, adenoma, adenocarcinoma, blastoma, sarcoma, or lymphoma.
